(19) United States
(12) Patent Application Publication
(10) Pub. No.: US 2021/0350932 A1
(43) Pub. Date: Nov. 11, 2021

(54) SYSTEMS AND METHODS FOR PERFORMING A GENOTYPE-BASED ANALYSIS OF AN INDIVIDUAL

(71) Applicant: The DNA Company Inc., Mississauga (CA)

(72) Inventors: Mansoor MOHAMMED, Oakville (CA); Kashif SIDDIQUI, Mississauga (CA); David LIEPERT, Calgary (CA); Kashif KHAN, Mississauga (CA)

(73) Assignee: The DNA Company Inc., Mississauga (CA)

(21) Appl. No.: 17/245,300

(22) Filed: Apr. 30, 2021

Related U.S. Application Data
(60) Provisional application No. 63/021,237, filed on May 7, 2020.

(51) Int. Cl. G16H 50/30; G16H 10/40; G06N 20/00

(52) U.S. Cl. CPC G16H 50/30; G06N 20/00; G16H 10/40

(57) ABSTRACT

Systems and method for performing a genotype-based analysis of an individual are discussed. An exemplary method may include: causing an interactive interface of an assessment application operating on a user device to prompt for responses to one or more phenotype interrogatories from an individual; receiving, from the user device, responses to the one or more phenotype interrogatories from the individual, entered via the interactive interface; using a relational model, determining at least one genotype classification for the individual based on the received responses to the one or more phenotype interrogatories; and causing the interactive interface to output information associated with the at least one genotype classification.

### RELATED APPLICATIONS

This application claims the benefit of priority to U.S. Provisional Application No. 63/021,237 entitled “SYSTEMS AND METHODS FOR DETERMINING SYMPTOM SEVERITY RISK AND INTERVENTION RECOMMENDATION,” the disclosure of which is incorporated in its entirety.

### TECHNICAL FIELD

Various embodiments of the present disclosure relate generally to determining a severity risk of symptoms of a disease, virus, or other illness, and intervention recommendations, and, in some embodiments, relate particularly to methods and systems for using a genotype-based model to relate responses for phenotyping questions to a symptom severity risk and intervention recommendation.

### BACKGROUND

Each patient afflicted with a disease, virus, or other physical or mental illness may experience it differently. While the illness may be commonly associated with one or more symptoms, each symptom may be more or less severe for each patient. A particular symptom may be mild or not even expressed in some patients, while severe or deadly in others. Susceptibility to certain symptoms has been associated with physiological conditions and/or genetic markers, but the relation between disease expression and physiology and/or genetics may not be well understood. Knowing whether a particular symptom is likely to be more mild or severe for a particular patient may assist with patient awareness and safety, interventions to promote patient health prior to contracting the illness, and/or treatment of the illness if and when contracted.

However, physiological and/or genetic testing may not be practical. For example, in the case of a global pandemic such as the COVID-19 outbreak, it may be impractical to perform tests requiring laboratory analysis on the general population due to, for example, testing availability, time constraints, cost, etc. Further, data provided by such tests may only be useful for assessing the potential severity of symptoms that already have an understood relation to physiology and/or genetics.

The present disclosure is directed to addressing one or more of these above-referenced challenges or other challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for performing a genotype-based analysis of an individual.

In one aspect, an exemplary embodiment of a method for performing a genotype-based analysis of an individual may include: causing an interactive interface of an assessment application operating on a user device to prompt for responses to one or more phenotype interrogatories from an individual; receiving, from the user device, responses to the one or more phenotype interrogatories from the individual, entered via the interactive interface; using a relational model, determining at least one genotype classification for the individual based on the received responses to the one or more phenotype interrogatories; and causing the interactive interface to output information associated with the at least one genotype classification.

In another aspect, an exemplary embodiment of a method for generating a relational model for performing a genotype-based analysis of an individual may include: inputting phenotype interrogatories responses from one or more individuals into a first machine-learning model as training data; inputting genotypes of the one or more individuals into the first machine-learning model as ground truth; and using the first machine-learning model to learn associations between the phenotype interrogatory responses and the genotypes, wherein the learned associations of the first machine-learning model include one or more different weights or groupings applied to the phenotype interrogatories responses, such that the learned associations of the first machine-learning model are usable to determine one or more genotype classification of an individual based on one or more phenotype interrogatory response from the individual.

In a further aspect, an exemplary embodiment of a system for performing a genotype-based analysis of an individual may include: a memory storing instructions and a relational model; and a processor operatively connected to the memory and configured to execute the instruction to perform operations. The operations may include: causing an interactive interface of an assessment application operating on a user device to prompt for responses to one or more phenotype interrogatories from an individual; receiving, from the user device, responses to the one or more phenotype interrogatories from the individual, entered via the interactive interface; using the relational model, determining at least one genotype classification for the individual based on the received responses to the one or more phenotype interrogatories; and causing the interactive interface to output information associated with the at least one genotype classification.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary embodiment of a computing environment that may be used to implement one or more aspects of the present disclosure.

FIG. 2 depicts an exemplary process for providing a health assessment for a user, according to one or more embodiments.

FIGS. 3A-3I depict exemplary user interface of an assessment tool for providing a health assessment for a user, according to one or more embodiments.

FIG. 4 depicts an example of a computing device, according to aspects of the present disclosure.

FIG. 5 depicts an exemplary method of performing a genotype-based analysis of an individual, according to one or more embodiments.

FIG. 6 depicts another exemplary method of performing a genotype-based analysis of an individual, according to one or more embodiments.

### DETAILED DESCRIPTION OF EMBODIMENTS

The terminology used in this disclosure is to be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. The following description is exemplary and explanatory only and is not restrictive of the features described with regard to any particular embodiment or example.

In this disclosure, the term “based on” means “based at least in part on.” The singular forms “a,” “an,” and “the” include plural referents unless the context dictates otherwise. The term “exemplary” is used in the sense of “example” rather than “ideal.” The term “or” is used disjunctively, such that “at least one of A or B” includes, (A), (B), (A and A), (A and B), etc. The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. Relative terms, such as, “substantially,” “approximately,” “about,” and “generally,” are used to indicate a possible variation of ±10% of a stated or understood value.

In this disclosure, the term “computer system” generally encompasses any device or combination of devices, each device having at least one processor that executes instructions from a memory medium. Additionally, a computer system may be included as a part of another computer system. An “electronic application” or the like generally encompasses a program, website, service, interface, or the like that may be accessed by a user via an electronic device such as a computer system like a desktop computer, mobile device, or the like in order to provide goods, services, information, or the like to the user.

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

As used herein, the term “provider” may indicate, and may be used interchangeably with, a doctor, hospital, medical insurance provider, health institution such as a governmental heath institution or regulatory entity, a source or vendor of medical items, services, or information, or the like. The term “user” may indicate, and may be used interchangeably with, a customer, patient, person with a medical issue and/or a person seeking information, treatment, intervention, or the like associated with a medical issue.

According to certain aspects of the disclosure, methods, systems, and non-transitory computer-readable media are disclosed for determining an illness symptom severity risk and intervention recommendation. Each of the examples disclosed herein may include one or more of the features described in connection with any of the other disclosed examples.

In general, the present disclosure provides methods and systems for determining a severity risk of one or more symptoms of an illness, prior to or after a person contracts the illness. Methods and systems according to this disclosure may assist with determining how severe one or more symptoms of an illness may be for a particular patient, if and when the patient contracts the disease. This determination may be a separate consideration to how at risk or likely the patient is to contract the disease, but in some cases these considerations may be related. The present disclosure also provides methods and systems for determining one or more intervention recommendations for the patient. An intervention recommendation may include a recommended action for the patient before and/or after the patient may have contracted the illness. As will be discussed below in more detail, in methods and systems according to the present disclosure, existing techniques may be improved.

When an illness is associated with symptoms that may be expressed over a wide range of severity, it may be beneficial for a patient and/or a medical provider to appreciate whether the patient is at risk of a severe expression of a symptom. For example, the patient experience when contracting the COVID-19 virus may range from asymptomatic to acute risk of mortality. Appreciation of such a risk prior to contracting a disease may enable an intervention that can one or more of increase the patient's overall health, decrease the severity of an otherwise severe symptom should the illness be contracted, or better inform the patient as to the risks associated with the illness. Appreciation of such a risk after the illness may have been contracted, but before symptoms may have fully progressed, may enable an intervention that is beneficial to the patient's care and/or health.

However, it may be difficult to determine whether a particular symptom is at risk of being severe for a particular patient. While some symptoms have been associated with particular genetic or physiologic qualities, such associations may not be well understood. Further, testing for the presence of such qualities in a particular patient may be impractical due to, for example, finite testing or laboratory resources, cost, timeliness, or the like. Such factors may be exacerbated in the face of a global pandemic, such as COVID-19.

Accordingly, a need exists to improve the understanding of the relation between symptoms of an illness and genetic or physiological qualities of a particular patient. A need also exists to improve the determination of whether a particular patient is at risk of experiencing a particular symptom with severity. A need additionally exists to improve recommendations for patient intervention on a per-symptom basis.

In embodiments, systems and methods of this disclosure may be used for any illness. Illness, throughout this disclosure, includes a disease, a virus, a cold or flu, a bacterial infection, an injury, a cancer, any chronic illness, any cardiovascular, gastrointestinal, or pulmonary disease, or any other mental or physical ailment, disorder, syndrome, or disability known now or in the future.

In an exemplary use case, a user and/or a medical provider for the user may desire to determine whether a particular symptom of an illness is likely to be more mild or severe for the user. The user and/or the provider may access an assessment tool, e.g. via an electronic application, a website page, or the like. The assessment tool may prompt the user to answer one or more preliminary questions, such as demographic questions, location questions, known-risk questions, urgent medical need questions, lifestyle questions, current health status questions, or the like, as discussed in further detail below. The assessment tool may provide the user with information about the illness, such as symptoms, contagion vectors, treatments, etc. The assessment tool may prompt the user for account information, privacy and/or medical information consent, or other authentication related information, as discussed in further detail below.

The assessment tool may also prompt the user to answer one or more phenotype questions. A phenotype question is a question having an answer that may be probative of whether the user exhibits a particular genotype, e.g., a gene or genetic marker associated with one or more physical or physiological traits. As discussed in further detail below, the answers to the phenotype questions and/or one or more genotypes of the user suggested by the answers may be indicative of how severe one or more symptoms of the illness may be for the user. In some embodiments, the answers to the phenotype questions and/or one or more genotypes of the user suggested by the answers may be probative of other medical information associated with the user such as, for example, sensitivity to environmental or medical factors, susceptibility to or presence of one or more illnesses, or the like.

The assessment tool may employ one or more relational models to the answers for the phenotype questions such as, for example, a first model relating the answers to the phenotype questions to one or more genotypes, a second model relating one or more genotypes to a severity risk assessment for one or more symptoms, a third model that directly relates the answers to the phenotype questions to the severity risk assessment, or the like. Using the one or more models, the assessment tool may determine a health assessment for the user. The health assessment may include, for example, a general risk assessment for how severe the illness may be for the user, a risk assessment for how severe one or more symptoms of the illness may be for the user, and one or more interventions for the user. Interventions may be directed toward the general health of the user, a particular symptom of the illness, a particular health issue of the user, or the like. Interventions may be an act to be performed by the user, a medication or supplement to be taken by the user, a therapy or medical provider recommendation or referral, or the like. The assessment tool may display the health assessment to the user and/or the medical provider.

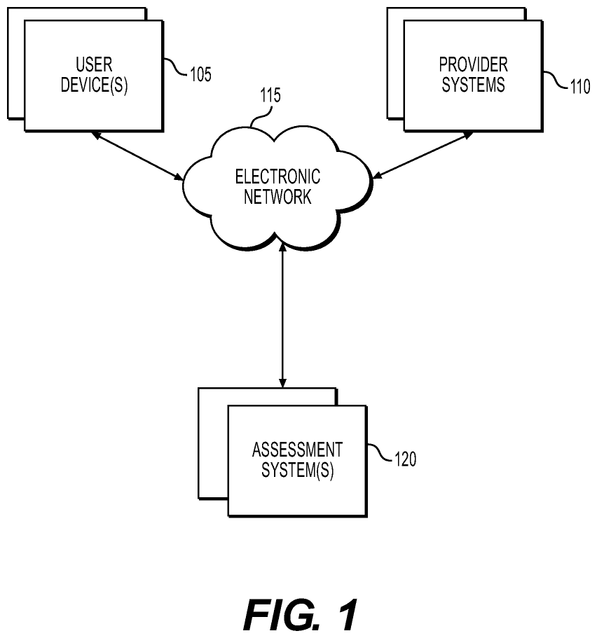

FIG. 1 depicts an exemplary client-server environment that may be utilized with techniques presented herein. One or more user device(s) 105 and/or one or more provider system(s) 110 may communicate across an electronic network 115. The systems of FIG. 1 may communicate in any arrangement. The user device 105 may be associated with a user, e.g., a person seeking information, treatment, intervention, or the like for a medical issue such as a disease the user does not yet have, a disease the user has already contracted, a pre-existing condition, a susceptibility to an ailment, or the like. The user device 105 may be operated by a user, of may be operated by a medical provider or other intermediary associated with the user.

As will be discussed herein, one or more assessment system(s) 120 may communicate with each other and/or with the user device 105 and/or the provider system 110 over the electronic network 115 in providing an assessment tool for determining a health assessment of the user. In some embodiments, the assessment tool may employ a machine learning model in order to, for example, develop, train, update, or reinforce a relational model, evaluate answers provided by the user to determine the health assessment, or the like. As used herein, a “machine learning model” may include data (e.g., illness symptom data, user medical data, phenotype data, genotype data, demographic data, or historical user data) or instruction(s) for generating, retrieving, and/or analyzing such data.

In various embodiments, the electronic network 115 may be a wide area network (“WAN”), a local area network (“LAN”), personal area network (“PAN”), or the like. In some embodiments, electronic network 115 includes the Internet, and information and data provided between various systems occurs online. “Online” may mean connecting to or accessing source data or information from a location remote from other devices or networks coupled to the internet. Alternatively, “online” may refer to connecting or accessing an electronic network (wired or wireless) via a mobile communications network or device. The Internet is a worldwide system of computer networks—a network of networks in which a party at one computer or other device connected to the network can obtain information from any other computer and communicate with parties of other computers or devices. The most widely used part of the Internet is the World Wide Web (often-abbreviated “WWW” or called “the Web”).

While FIG. 1 depicts the various systems as physically separate and communicating across network 115, in various embodiments features of certain systems, such as the assessment system 120, may be incorporated partially or completely into any of the other systems of FIG. 1. For example, the assessment system 120 may be incorporated by the provider system 110 and/or the user device 105. In another example, the user device 105 may be incorporated into the provider system 110, or vice versa. In some embodiments, some or all of the functionality of the machine learning model may be incorporated into the user device 105. In some embodiments, some or all of the functionality of the assessment system 120 may be incorporated into an electronic application or website accessible by the user device 105.

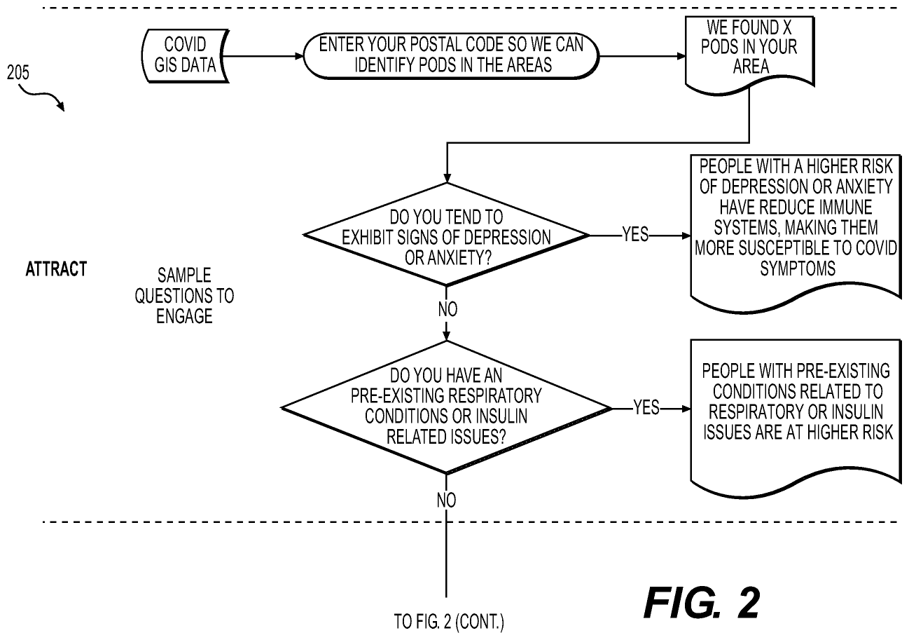

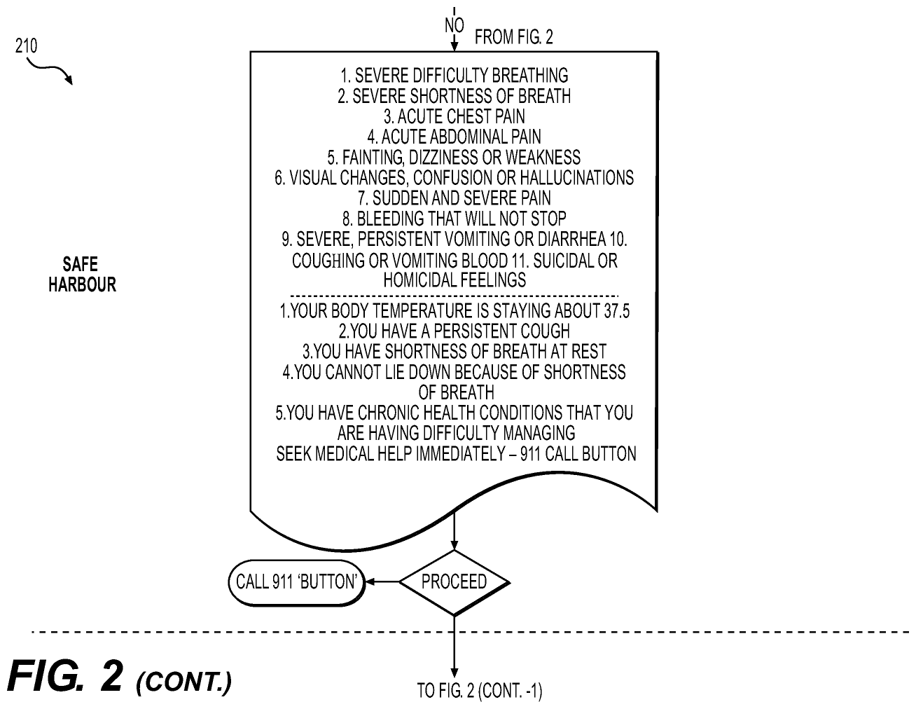

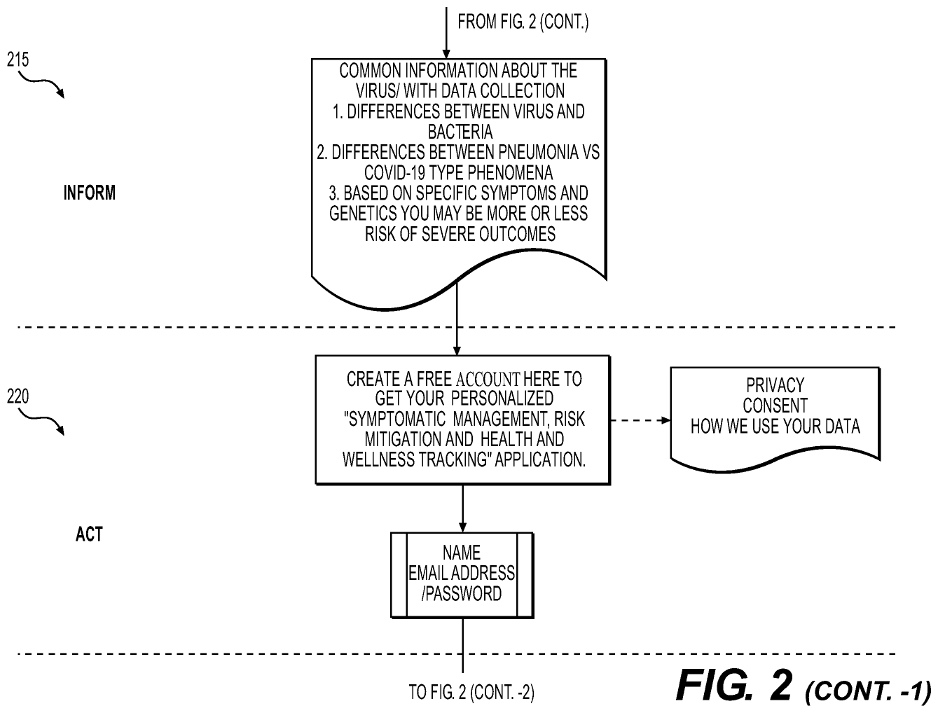

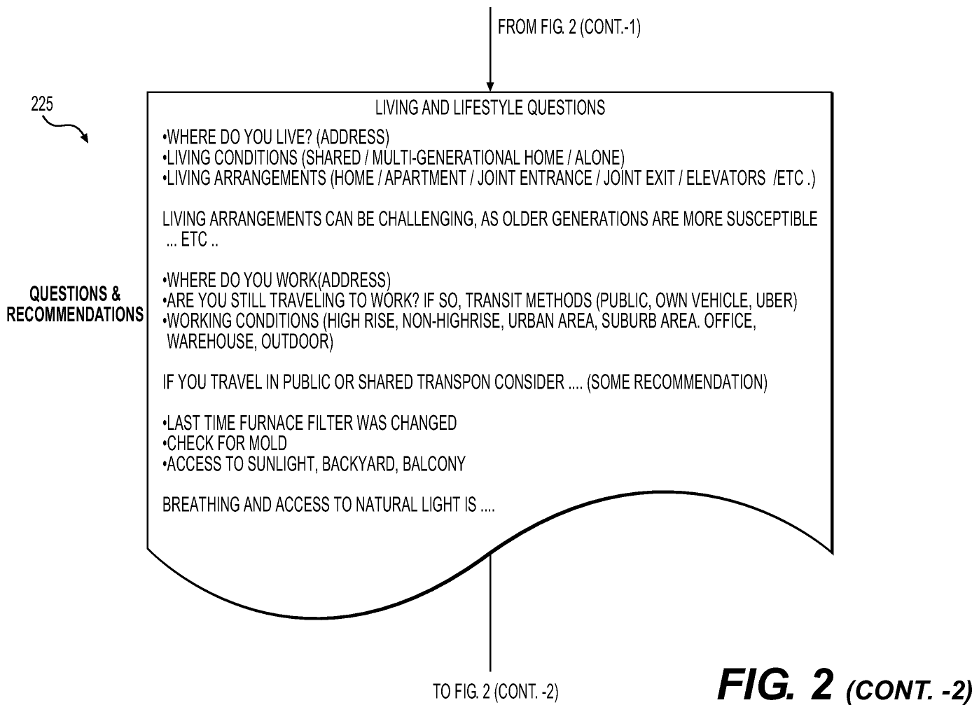

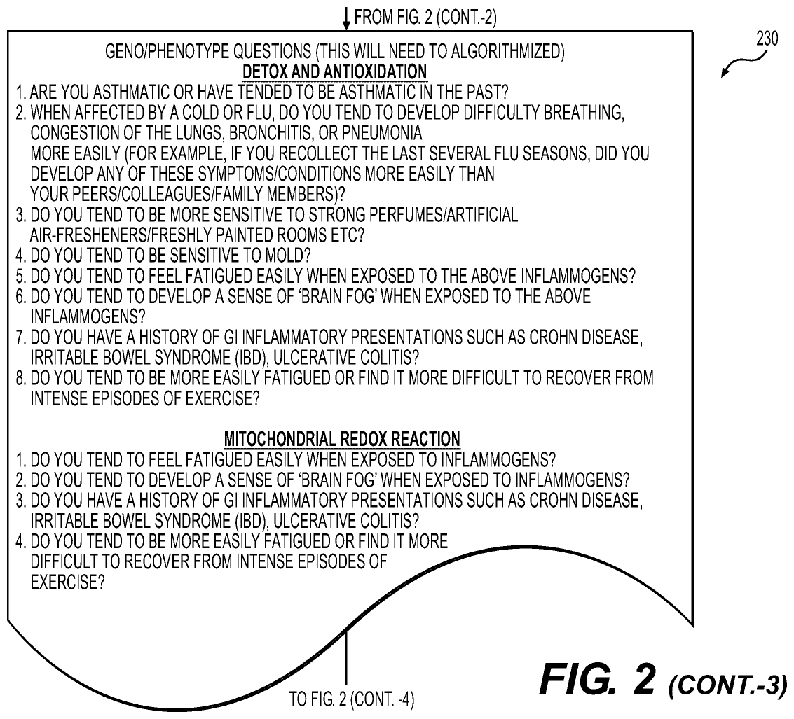

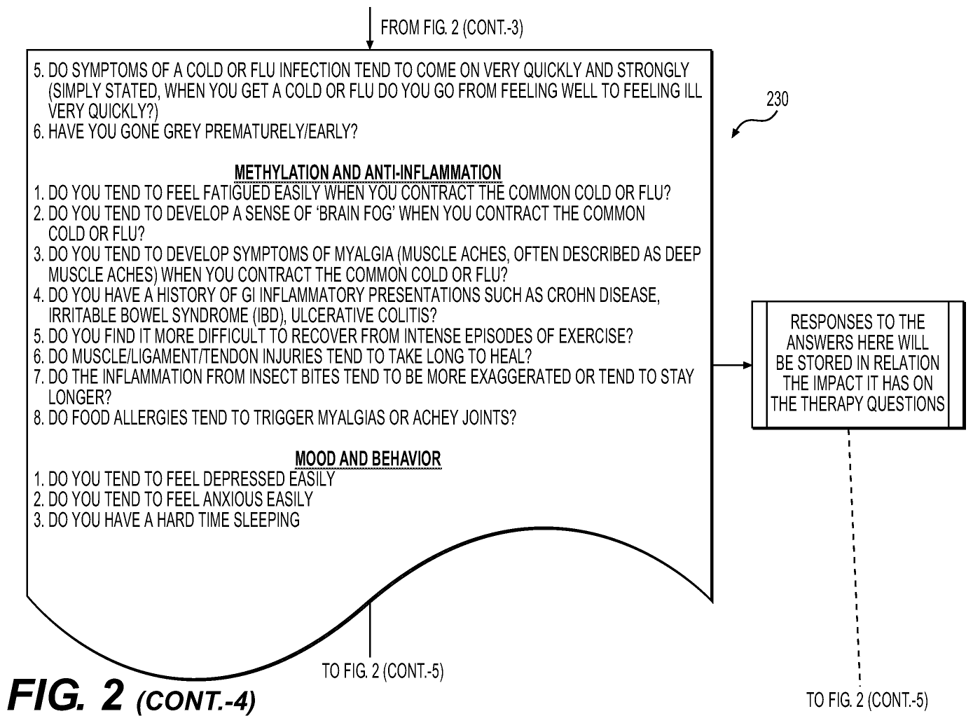

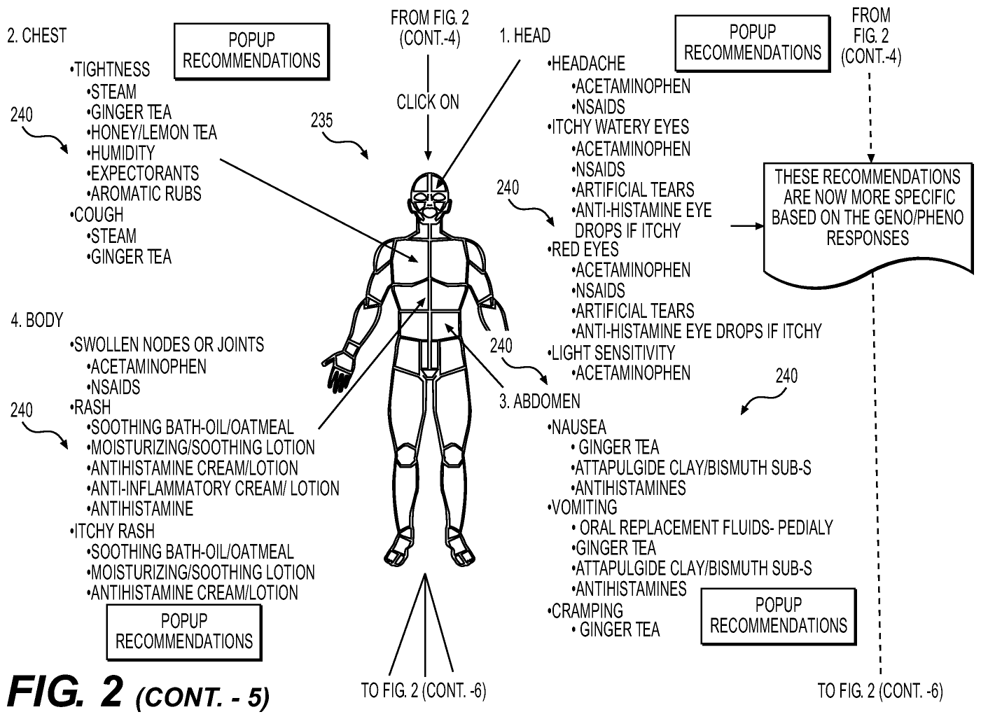

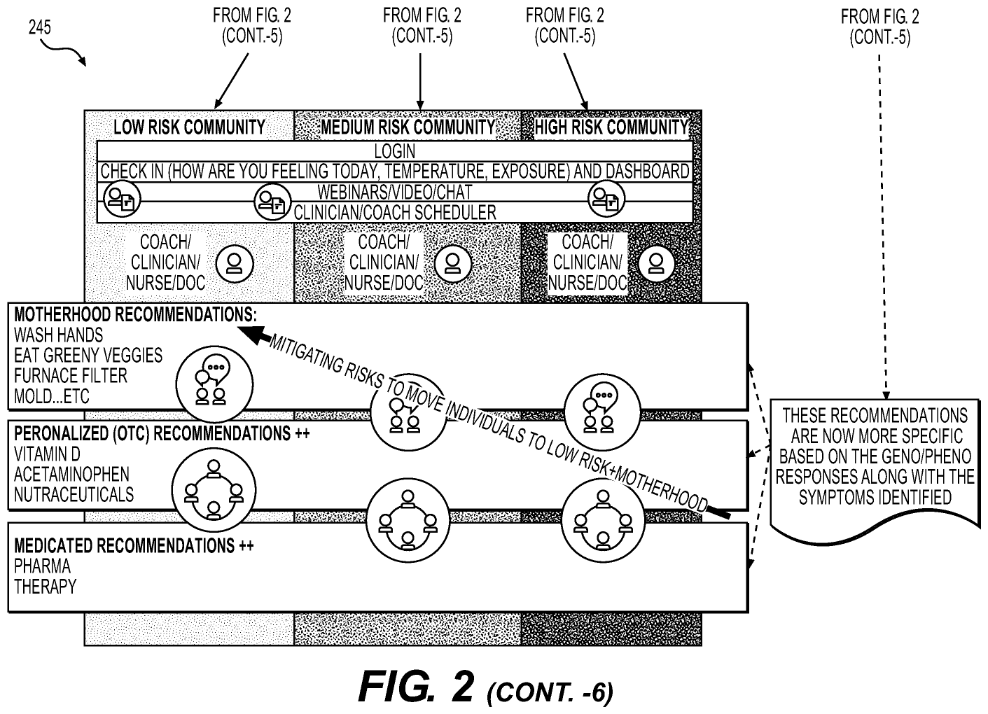

FIG. 2 illustrates an exemplary process for providing a heath assessment to a user. A user and/or a medical provider desiring a health assessment for the user, and in particular a symptom severity risk assessment and intervention recommendation for a particular illness, may access an assessment tool provided by the assessment system 120, e.g., via a user device 105 or a provider system 110, or the like. The assessment tool may employ an electronic application, a software program, a website page, or the like. It should be understood that, in various embodiments, actions described as performed by the assessment tool may be performed by, for example, the assessment system 120, the user device 105, the provider system 110, another system, or combinations thereof.

At step 205, the assessment tool may prompt the user to answer one or more preliminary questions. In various embodiments, the preliminary questions may include, for example, non-personally identifying questions such as, for example, demographic questions, location questions, known-risk questions, or the like that are usable, for example, to establish a baseline profile for the user using non-personally identifying data. Known risk questions may include, for example, general health questions that may be probative to an increased severity to symptoms generally, such as whether the user exhibits signs of anxiety or depression or other signs of conditions known to reduce immune system performance or exacerbate symptoms. Known risk questions may include whether the user has a pre-existing condition or another medical issue like an insulin related issue or respiratory issue known to be associated with symptoms associated with the illness.

In some embodiments, in conjunction with prompting for answers to the preliminary questions, the assessment tool may provide information related to answers received for the preliminary questions. For example, the assessment tool may identify whether and how many clusters of pods of individuals known to have contracted the illness based on location data associated with the user. The assessment tool may provide information associated with known risks and/or how such risks are associated with symptoms of the illness.

In some embodiments, instead of or in addition to prompting for answers for the preliminary questions, the assessment tool may retrieve information associated with the preliminary questions from one or more sources. For example, the assessment tool may retrieve information from the user device such as location information, information from a provider system 110 such as profile or medical information associated with the user, or information from an online source such as disease spread, symptom, or treatment information. In some embodiments, such retrieval is performed in response to receiving user approval, a user login request, an authentication of the user, or the like, as discussed in more detail below.

At step 210, the assessment tool may prompt for answers to one or more safe-harbor questions. A safe-harbor question is a question having an answer that may be probative of whether the user has an immediate need for emergency medical intervention. In response to a positive answer to one or more of the safe-harbor questions, the assessment tool may provide an indication that the user should seek immediate medical assistance. In some embodiments, the indication may be associated with a call to action such as a button or the like selectable by the user to contact medical assistance such as 911 emergency services. In some embodiments, the assessment tool may automatically perform such contact.

At step 215, the assessment tool may provide the user, e.g., via the user device 105 or another system, information associated with the illness. Information associated with the illness may include, for example, symptoms, contagion vectors, treatments, whether the illness is viral or bacterial, differences or similarities between the illness and another illness or illnesses, a message, or the like. A message may include a statement such as that based on specific symptoms, genetics, or other factors, the user may be more or less at risk of severe outcomes, or that the user may be more or less at risk of a particular symptom being severe based on genetics, environment, or other factors.

At step 220, the assessment tool may issue a prompt for user account information. Such a prompt may include a request for existing account information, a request for consent to use personally identifying information and/or medical information associated with the user, a prompt to register a new account, a prompt for authenticating the user, information related to information privacy and data use, or the like. In some embodiments, in response to information and/or consent received at step 220, the assessment tool may retrieve user profile information and/or user medical information from at least one source such as, for example, the provider system 110, a memory of the user device 105, a memory of the assessment system 120, a social information source like a social media profile of the user, or the like. User profile information may include descriptive information associated with the user such as age, gender, location, marital status, family and/or social connections, employment, hobbies, activities, travel history, etc. User medical information may include medical history data, medical test or diagnostic data, physiological or genetic data, or the like.

At step 225, the assessment tool may prompt for answers to lifestyle questions. A lifestyle question is a question having an answer that may be probative about an environment or condition that the user may be exposed to. In some embodiments, the assessment tool may provide general information and/or recommendations based on positive responses to one or more of the lifestyle questions.

At step 230, the assessment tool may prompt for answers to a plurality of phenotype questions. In some embodiments, the plurality of phenotype questions may be separated into clusters. Clusters may be based on, for example, a genotype related to one or more phenotypes associated with the phenotype questions in the cluster, a particular symptom or category of symptoms, a particular physiology or category of physiology, or the like.

At step 235, the assessment tool may prompt for identification of one or more locations on the user's body that are currently experiencing discomfort and/or a symptom, and/or one or more locations on the user's body for which the user may desire information related to a risk of severity of a symptom, susceptibility to an illness, or the like. In the embodiment depicted in FIG. 2, the prompt for step 235 may include an illustration of a human body having selectable locations. In some embodiments, in response to selection of a location on the body, the assessment tool may prompt for information such as, for example, a type of discomfort or illness currently experienced, a level of pain experienced at that location, a symptom of interest associated with that location, or the like.

In some embodiments, the prompt at step 235 is performed at multiple occasions in order to track one or more discomforts or symptoms over time. In some embodiments, the intervention recommendation is based on trends, patterns, or changes in the one or more discomforts/symptoms over time.

At step 240, the assessment tool may provide one or more intervention recommendations associated with a selected location. In some embodiments, the intervention recommendation is based on the information received in response to the prompt for information in step 235. In some embodiments, the intervention recommendation may be based on the answers to one or more of the preliminary questions, the phenotype questions, or other information received or retrieved by the assessment tool. In some embodiments, the intervention recommendation may be based on a health assessment for the user, as discussed in further detail below.

In some embodiments, one or more intervention recommendations may be associated with each discomfort/symptom, and one or more discomforts/symptoms may be associated with each location. In some embodiments, the one or more intervention recommendations provided by the assessment tool for a selected location may be based on a summation of intervention recommendations for the discomforts/symptoms associated with that location. For example, a location, such as the head, may have a discomfort of headache associated with an intervention of Acetaminophen and/or NSAIDs, and a discomfort of light sensitivity associated with an intervention of Acetaminophen NSAIDs, artificial tears, and/or anti-histamine eye drops. The assessment tool may sum the interventions for the discomforts/symptoms, and provide an intervention recommendation of Acetaminophen and NSAIDs, since those interventions had the highest sum of listings for the discomforts for the location of the head.

In some embodiments, the assessment tool includes prompts for the following locations and discomforts/symptoms, which are associated with the following interventions, although it should be understood that other and other combinations of locations, discomforts, symptoms, and interventions may be included on other embodiments.

Head
Headache
   Acetaminophen
   NSAIDS
itchy watery eyes
   Acetaminophen
   NSAIDS
   Artificial Tears
   Anti-histamine eye drops if itchy
Red eyes
   Acetaminophen
   NSAIDS
   Artificial Tears
   Anti-histamine eye drops if itchy
Light Sensitivity
   Acetaminophen
   NSAIDS
   Artificial Tears
   Anti-histamine eye drops if itchy
a Sore Throat
   Salt-water gargle-do not swallow
   Dilute Hydrogen Peroxide Gargle-do not swallow
   Honey/Lemon Tea
   Ginger Tea
   Throat Lozenges
      Zinc
      Local Anesthetic
      Antiseptic
   Acetaminophen
   NSAIDS
Scratchy Throat
   Salt-water gargle-do not swallow
   Dilute Hydrogen Peroxide Gargle-do not swallow
   Honey/Lemon Tea
   Ginger Tea
   Throat Lozenges
      Zinc
      Local Anesthetic
      Antiseptic
   Acetaminophen
   NSAIDS
Runny Nose
   Nasal Rinses
   Double-dilute Hydrogen Peroxide spray/drops
   Antihistamines
   Decongestants
   Expectorants
Congested Nose
   Nasal Rinses
   Double-dilute Hydrogen Peroxide spray/drops
   Antihistamines
   Decongestants
   Expectorants
   NAC (max 600 mg/day) plus Vitamin C (max 1 gm/day)
Lost sense of smell
   Nasal Rinses
   Double-dilute Hydrogen Peroxide spray/drops
   Antihistamines
   Decongestants
   Expectorant
   NAC (max 600 mg/day) plus Vitamin C (max 1 gm/day)

Chest
Tightness
   Steam
   Ginger Tea
   Honey/Lemon Tea
   Humidity
   Expectorants
   Aromatic Rubs
   NAC (max 600 mg/day) plus Vitamin C (max 1 gm/day) Cough
Steam
   Ginger Tea
   Honey/Lemon Tea
   Humidity
   Anti-tussives/cough suppressants
   Expectorants
   Aromatic Rubs
   NAC (max 600 mg/day) plus Vitamin C (max 1 gm/day)

Abdomen
Nausea
   Ginger Tea
   Attapulgide Clay/Bismuth Sub-salicylate
   Antihistamines
Vomiting
   Oral replacement fluids-Pedialyte/Gatorade
   Ginger Tea
   Attapulgide Clay/Bismuth Sub-salicylate
   Antihistamines
Cramping
   Ginger Tea
   Attapulaide Clay/Bismuth Sub-Salicylate
   Anticholinergics
   Antihistamines
   Acetaminophen
Diarrhoea
   Oral replacement fluids-Pedialyte/Gatorade
   Attapulgide Clay/Bismuth Sub-Salicylate
   Loperamide

Body
Swollen lymph nodes or joints
   Acetaminophen
   NSAIDS
Rash
   Soothing bath-Oil/Oatmeal
   Moisturizing/soothing lotion
   Antihistamine cream/lotion
   Anti-inflammatory cream/lotion
   Antihistamine
Itchy Rash
   Soothing bath-Oil/Oatmeal
   Moisturizing/soothing lotion
   Antihistamine cream/lotion
   Anti-inflammatory cream/lotion
   Antihistamine
Fever
   Rest
   Hydration
   Acetaminophen
   NSAIDS
   Back Cumin (N. sativa as oil maximum 3 tsp/day)
   Thunder-God Vine (T. wilfordii maximum 350 mg/day)
   Turmeric (C. longa maximum 500 mg/day)
Lethargy
   Rest
   Hydration
   Acetaminophen
   NSAIDS
   Black Cumin (N. sativa as oil maximum 3 tsp/day)
   Thunder-God Vine (T. wilfordii maximum 350 mg/day)
   Turmeric (C. longa maximum 500 mg/day)
Malaise
   Rest
   Hydration
   Acetaminophen
   NSAIDS
   Black Cumin (N. sativa as oil maximum 3 tsp/day)
   Thunder-God Vine (T. wilfordii maximum 350 mg/day)
   Turmeric (C. longa maximum 500 mg/day)
Myalgias
   Rest
   Hydration
   Magnesium
   Ginger Tea
   Acetaminophen
   NSAIDS
   Black Cumin (N. sativa as oil maximum 3 tsp/day)
   Thunder-God Vine (T. wilfordii maximum 350 mg/day)
   Turmeric (C. longa maximum 500 mg/day)

At step 245, the assessment tool may determine a health assessment for the user. The health assessment may be based on any of the information received or retrieved by the assessment tool, and in particular may be based on the answers to the phenotype questions. The assessment tool may employ one or more relational models to analyze and/or interpret the data. In some embodiments, the one or more relational models may include predetermined relationships between answers to the phenotype questions and one or more of a genotype, an illness, a symptom, or combinations thereof. In some embodiments, the one or more relational models may include one or more machine learning models. In some embodiments, the relational model is initialized with predetermined relationships, and then is tuned, updated, or modified based on additional data. In some embodiments, such tuning, updating, or modifying is performed by employing one or more machine learning techniques. Various examples of relational models are discussed in further detail below.

The health assessment may include, for example, a severity risk assessment for the user. In some embodiments, the severity risk assessment may include an overall severity risk for the user for the illness as a whole. In some embodiments, the severity risk assessment may include a severity risk assessment for one or more symptoms of the illness. In some embodiments, determining the health assessment may include determining whether the user falls into a high risk category, a medium risk category, or a low risk category, and the health assessment may include an indication of the determined category such as, for example, a label, a color along a gradient, or any other suitable indication. In some embodiments, the category may be applied on a per-symptom basis. In some embodiments, the category may be applied to a disease in general. In some embodiments, the assessment tool may select and/or prioritize the one or more intervention recommendations based on the determined category of risk for the user. In some embodiments, the assessment tool may select and/or prioritize the one or more intervention recommendations based on a criteria such as, for example, cost, difficulty, frequency of need, need for professional medical intervention, side-effect risk, or any other suitable criteria.

The health assessment may include one or more intervention recommendations for the user. In some embodiments, the one or more intervention recommendations are directed toward one or more of a general health improvement of the user, an illness as a whole, or a particular symptom of an illness. In some embodiments, the one or more intervention recommendations may be clustered based on, for example, the severity risk assessment, the type of intervention (e.g., lifestyle, over-the-counter, prescription, therapeutic, etc.), combinations thereof, of the like. In some embodiments, the health assessment is configured to act as a portal for the user to track his/her health status, contact a health professional, view information that may be relevant based on the intervention recommendations for the user, or the like.

In some embodiments, the health assessment is configured to be updated and/or to track the user's health over time. In some embodiments, the health assessment is configured, such as by selection and/or prioritization of intervention recommendations, to cause the user to migrate from a higher risk category of a symptom or of the disease to a lower risk category, and/or to migrate from intervention recommendations requiring professional medical intervention like a prescription or therapy, to over-the-counter interventions and/or lifestyle or wellness recommendations.

As discussed above, the assessment tool may employ one or more relational models to determine one or more intervention recommendations and/or severity risk assessments for the user. Different relational models may be associated with different illnesses, symptoms, genotypes, demographics, or combinations thereof.

In an example, one or more relational models may be associated with the COVID-19 virus. The COVID-19 pandemic is caused by a strain of coronavirus known as the SARS-Co-V2 virus. This virus is from the same family of viruses that caused both the SARS and MERS outbreaks several years ago. These viruses appear to originate from animals, such as bats, and are ultimately passed on to humans. The SARS-Co-V2 virus, like similar strains before it, enters the human cell via the ACE2 receptor. The ACE2 gene that encodes this receptor is found on the X chromosome and should not be confused with the ACE1 gene found on chromosome 17. The ACE2 receptor is expressed on cells of the lower respiratory tract as well as cells of the cardiovascular and renal (kidney) system.

For the majority of individuals (85-90%) infected with the SARS-Co-V2 virus, it appears that the symptoms and course of the infection will be no more severe than the common flu. However, the mortality rate of this virus (˜3-5%) appears higher than the common flu, and up to 10% of individuals infected with this virus may need acute hospital care. Mercifully, in the absence of severe pre-existing health concerns, the virus does not appear to result in serious complications in children. The SARS-Co-V2 virus appears to be able to survive airborne for about 3 hours, but much longer on solid surfaces (up to 3-5 days).

Importantly, the virus seems to (i) shed at an alarming rate (i.e. how many viruses are replicated and released from an infected human cell—early studies suggest up to 1000× more than previous coronaviruses), and (ii) shed very early in the infection cycle—often before even moderate symptoms develop. These two features, coupled with the approximately 10% of the infected individuals that may need emergency care, constitute another danger of this virus—the ability to cripple hospital and healthcare systems.

Important health considerations to consider during the COVID-19 pandemic are: (i) factors for or against overall health and vitality; (ii) factors for or against the optimal performance of the immune system; and (iii) factors for or against the ability to deal with acute inflammatory responses. It should be noted that the majority of severe cases of COVID-19 appear related to cytokine storms that acutely damage the lining of the lungs and potentially that of the cardiovascular and renal systems (not unlike what happens during septic shock).

Genomic predispositions that individualize points (i)-(iii) include:
Possible/likely correlation to age, with increased risk of complications in elder populations. While it is difficult to define elder in this context, it would be conservative to be more cautious in individuals older than 70.
Possible sex preponderance. Epidemiologic data from multiple countries suggest the following trend: Among gender-identified disease cases, men make up 60 percent of those that progress to the dangerous pneumonia stage. They make up 59 percent of the hospitalizations, 72 percent of the intensive care unit admissions and 65 percent of the deaths.

COVID-19 has also been associated with several symptoms and effects, several of which are discussed below.

Vitamin D Activation, Transport, and Uptake

One of the results of the COVID-19 pandemic is wide-spread stay-at-home orders and quarantining. One of the immediate and concerning ripple effects of reduced mobility and self-isolation is exposure to sunlight. Individuals with genetic predisposition unfavorable to vitamin D utilization may experience more severe deficiency in vitamin D. Vitamin D is responsible for, or contributes to, many cellular processes. Optimal levels of vitamin D are needed for the optimal function of the immune system, optimal mood and behavior, to normalize blood pressure and blood sugar, and aiding in anti-inflammatory capacity—all of which are important in the context of other symptoms associated with COVID-19. Vitamin D has been well-studied as a co-treatment for pneumonia and for moderating the cytokine storms (dangerous hyper-immune responses) associated with certain infections, including COVID-19.

As discussed above, the assessment tool may employ one or more relational models to relate answers to phenotype questions to one or more of genotypes, symptoms, or intervention recommendations. In some embodiments, the assessment tool employs a first relational model that relates a symptom severity risk (e.g., vitamin D deficiency) with genotypes. In some embodiments, the first relational model includes a predetermined list of genotypes associated with the symptom severity risk. In an exemplary embodiment, the following genetic markers (genes and genotypes) are associated with vitamin D utilization:

CYP2R1

RS10741657
AA: predisposed to optimal enzyme activity with optimal vitamin D3 production
AG: predisposed to suboptimal enzyme activity with suboptimal D3 production
GG: predisposed to suboptimal enzyme activity with suboptimal D3 production

VDBP/GC

RS4588
CC: predisposed to optimal vitamin D3 transportation and serum levels
CA: predisposed to suboptimal vitamin D3 transportation and serum levels
AA: predisposed to suboptimal vitamin D3 transportation and serum levels

VDR

RS1544410
CC: predisposed to optimal vitamin D3 binding and cellular uptake
CT: predisposed to suboptimal vitamin D3 binding and cellular uptake
TT: predisposed to suboptimal vitamin D3 binding and cellular uptake

Individuals with the G allele of CYP2R1, coupled with the A allele of VDBP and the T allele of VDR, when placed in an environment of poor sun exposure are at extreme risk of clinically deficient levels of vitamin D3. In this manner, the severity risk of vitamin D deficiency is linked to particular genotypes.

It should be understood that the first relational model above is exemplary only, and that in various embodiments, the first relational model may include various genotypes, genes, and/or genetic markers associated with various symptoms and/or illnesses. In some embodiments, the first relational model may include weights for particular genes or expressions. For example, for the VDR RS1544410 gene, a CT expression may be associated with a different severity of symptom than a TT expression, and the weights associated with the expressions may be indicative of such severity. Alternatively or additionally, weights may be indicative of other various factors such as prevalence in the population, confidence in association with the symptom and/or illness, or any other suitable criteria. Such weights may affect the determination of the health assessment of a user, as discussed in further detail below. In some embodiments, the weights may be predetermined. In some embodiments, the weights may be determined based on tracked symptom data and associated genetic data, e.g., via the use of a machine learning model.

In some embodiments, a second relational model between genotypes and phenotypes may include one or more phenotype questions predetermined to be associated with one or more genotypes. In an exemplary embodiment, one or more of the following phenotype questions may be included in the assessment tool, and may be probative of the vitamin D genotypes listed above from the first relational model:
1. Do you currently have from low serum vitamin D (that is, have recent serum vitamin D tests confirm that your levels are low or insufficient)?
2. Do you suffer from osteopenia or osteoarthritis?
3. Do you suffer from seasonal mood disorder (or in general feel less energized, less motivated, more depressed during winter months/gloomy low-sunlight days)?
4. Do you notice that you feel noticeably better (mood, energy, sense of well-being) when you get adequate sun exposure?
5. Do you notice that you feel noticeably worse (mood, energy, sense of well-being) when you get inadequate sun exposure?
6. Do you feel better when you take a vitamin D supplement?
7. Have you ever been diagnosed with a skin cancer or skin condition that precludes sun exposure?
8. Do you live in a geography/city/town/country with four seasons or at least with long periods of poor/low sunlight?
9. Are you a shift worker with days/weeks months during the year working during the night and sleeping during the day?
10. Do you tend to get the common cold/flu more frequently than others?
11. Do your colds/flus tend to develop into bronchitis and/or pneumonia?
12. Do you take a vitamin D supplement?
13. Does your home or living environment permit you to get sun exposure while indoors (adequate windows and directional exposure allowing sun exposure)?
14. Do you try to go for walks during the day even if only for brief periods?

Thus, the phenotypes corresponding to the questions above may be associated with the genotypes listed above for a risk of vitamin D deficiency. In some embodiments, multiple questions may be associated with the same phenotype and/or genotype. Genotypes may be expressed in different patients in different ways.

It should be understood that the second relational model above is exemplary only, and that in various embodiments, various phenotype questions may be associated with various genotypes for various symptoms and illnesses. In some embodiments, one or more of the phenotype questions may be associated with a weight. The weight may be indicative of one or more of, for example, a probative value of the question to one or more genotypes, a prevalence of the phenotype or associated genotype in the population, or the like. In some embodiments, the weights may be predetermined. In some embodiments, the weights may be determined based on tracked symptom data and answers to the questions from other users, such as via a machine learning model.

In some embodiments, the assessment tool may employ a third relational model that includes a predetermined set of rules that may be applied to the answers to the phenotype questions of the first relational model in order to determine one or more of a severity risk assessment or an intervention recommendation for a particular symptom. In this manner, the phenotypes from the second relational model may be related to the severity risk assessment and/or intervention recommendation. In an exemplary embodiment, the predetermined set of rules for the third relational model may include the following rules:

1. If at least one of questions 1-3 above has a yes/I don't know response, trigger an intervention recommendation for vitamin D; and

2. If at least three of questions 4-11 above have a yes/I don't know response OR at least one of questions 12-14 has a no response, trigger the intervention recommendation for vitamin D.

It should be understood that the third relational model above is exemplary only, and that in various embodiments, the third relational model may include various rules to be applied to various questions, clusters of questions, or the like. In some embodiments, the assessment tool may generate rules for the third relational model based on the weights from one or more of the first or second relational models. In some embodiments, the assessment tool may cluster one or more phenotype questions based on a criteria, such as by symptom, genotype, or the like. In some embodiments, the assessment tool may generate rules for the third relational model based on tracked symptom data. For example, the assessment tool may determine correlations between phenotype question answers and the tracked symptom data. In some embodiments, the comparison may also be based on one or more of the first or second relational models. In some embodiments, the comparison may employ a machine learning model.

The intervention recommendation for vitamin D may include a disclaimer, an action, and/or textual information. In an exemplary embodiment, the disclaimer for the vitamin D intervention may include the statement “Without appropriate genomic confirmation, we can only approximate your relative risk for genetic traits that impact your immune status and COVID-19 vulnerability through Vitamin D.” The action may include taking a dosage of vitamin D per day, e.g., 2000 IU. The textual information may include the statement “Interventions of potential benefit to you include Vitamin D3 supplementation (maximum 2000 IU/day), and maximizing your safe-sun exposure, particularly in the winter months.” In some embodiments, the textual information may additionally include a further statement with additional information, such as “Did you know that vitamin D is no longer even considered a vitamin? The way that your body makes, transports and responds to vitamin D means it is more like a hormone. Regardless, it is responsible for or contributes to innumerable cellular processes. Optimal levels of vitamin D are needed for the optimal function of your immune system, optimal mood and behavior modulation, normalizing your blood pressure and blood sugar, and aiding in your anti-inflammatory capacity—all of which are important in the context of the current COVID-19 pandemic. Vitamin D has been well-studied in the co-treatment of pneumonia and for moderating the cytokine storms—dangerous hyper-immune responses associated with certain infections, including COVID-19.”

Hypertension and Insulin Resistance

Initial epidemiologic data appear to suggest that two of the more significant comorbidities associated with poor COVID-19 outcomes (especially when combined with age) are pre-existing hypertension and insulin resistance/Type II diabetes. It should be understood that these pre-existing conditions may not be related with an increase in the risk of contracting COVID-19, but might increase the risk of severity of one or more symptoms. Moreover, it should be understood that initial suggestions linking increased risk of infections in patients on current ACE inhibitors or angiotensin receptor blockers (ARBs) may not yet have been validated. Regardless, innate genomic factors when combined with diet and lifestyle may be strong predictors of both hypertension and insulin resistance. While it is unclear if or why hypertension and insulin resistance/Type II diabetes might be associated with poorer COVID-19 outcomes, these conditions may impact optimal health. Anything that does so may diminish immune system response or exaggerate the pro-inflammatory effects of COVID-19.

In some embodiments, the first relational model includes an association between the following genetic markers (genes and genotypes) and hypertension and/or insulin resistance:

ACE

RS4343
GG: predisposed to increased angiotensin converting enzyme activity
AG: predisposed to increased angiotensin converting enzyme activity
AA: predisposed to normal angiotensin converting enzyme activity

ACE

RS4343
GG: increased predisposition to hypertension with a high fat diet
AG: increased predisposition to hypertension with a high fat diet
AA: normal predisposition to hypertension with a high fat diet

ACE

RS4343
GG: increased predisposition to insulin resistance with a high fat diet
AG: increased predisposition to insulin resistance with a high fat diet
AA: normal predisposition to insulin resistance with a high fat diet

CLOCK

RS1801260
CC: delayed and reduced sleep patterns with increased risk of hypertension and insulin resistance
TC: predisposition to normal sleep patterns with normal risk of hypertension and insulin resistance
TT: predisposition to normal sleep patterns with normal risk of hypertension and insulin resistance

TCF7L2

RS12255372
TT: increased risk of insulin resistance
GT: increased risk of insulin resistance
GG: normal risk of insulin resistance

CRY1

RS2287161
CC: increased risk of insulin resistance when combined with a high carb diet
GC: normal risk of insulin resistance. Dietary and lifestyle factors are more relevant
GG: normal risk of insulin resistance. Dietary and lifestyle factors are more relevant

DIO2

RS225014
CC: increased risk of insulin resistance when combined with a high carb diet
TT: normal risk of insulin resistance. Dietary and lifestyle factors are more relevant
TT: normal risk of insulin resistance. Dietary and lifestyle factors are more relevant

SLC30A8

RS11558471
AA: increased risk of elevated blood glucose. Benefits from increased zinc (14 mg/daily)
GA: increased risk of elevated blood glucose. Benefits from increased zinc (14 mg/daily)
GG: normal risk of elevated blood glucose. No additional benefit from increased zinc

In an exemplary embodiment, the following phenotype questions may be included in the second relational model as probative of the hypertension and insulin resistance genotypes listed above from the first relational model:
1. Are you currently or have previously suffered from insulin resistance and/or Type II diabetes?
2. Are you currently on any medications to normalize your blood sugars?
3. Has a healthcare provider ever flagged or raised a concern over your blood glucose levels; fasting blood glucose levels; HbA1C levels; insulin levels?
4. Are you currently or have previously suffered from hypertension (high blood pressure)?
5. Are you currently or have previously been on any medications for high blood pressure?
6. Has a healthcare provider ever flagged or raised a concern over your blood pressure?
7. Is your blood pressure highly responsive to dietary salts—have you been told by a healthcare provider to reduce your salt intake in order to reduce your blood pressure?
8. Do you tend to feel fatigued or jittery or lightheaded if you skip a meal or in general if you stay hungry past your normal eating habits?
9. Do you tend to feel fatigued or sleepy within 30-60 mins of eating a high carb meal?
10. Do you tend to feel fatigued or sleepy within 30-60 mins of eating a high fat meal?
11. Do you tend to crave something sweet within 30-60 mins of eating a high fat or high carb meal?
12. Do you tend to prefer high simple carb meals—white bread, pasta, potatoes, white rice?
13. Is your blood pressure highly responsive to dietary salts—have you been told by a healthcare provider to reduce your salt intake in order to reduce your blood pressure?
14. Do you tend to put on weight easily when you increase your carb intake?
15. Do you tend to lose weight easily when you decrease your carb intake?
16. Do you tend to put on weight easily when you increase your fat intake?
17. Do you tend to lose weight easily when you decrease your fat intake?
18. Do you tend to noticeably feel better, think better, be more productive when you purposefully reduce your intake of processed sugars—including sweets, sodas and fruit juices?
19. Have you ever noticed that high sugar foods/desserts/drinks trigger muscle and/or joint aches?

In an exemplary embodiment, the predetermined set of rules for the third relational model may include the following rules related to the hypertension and insulin resistance phenotype questions from the second relational model:

1. If at least one of questions 1-7 above has a yes/I don't know response, trigger an intervention recommendation for hypertension and insulin resistance; and

2. If at least five of questions 8-19 above have a yes/I don't know response, trigger the intervention recommendation for hypertension and insulin resistance.

The intervention recommendation for hypertension and insulin resistance may include a disclaimer, an action, and/or textual information. In an exemplary embodiment, the disclaimer for the hypertension and insulin resistance intervention may include the statement “Without appropriate genomic confirmation, we can only approximate your relative risk for genetic traits that impact your immune status and COVID-19 vulnerability through Hypertension and Insulin Resistance.” The action may include exercise and activity. The textual information may include the statement “Optimal exercise and activity.” In some embodiments, the textual information may additionally include a further statement with additional information, such as “Interventions of potential benefit to you include optimal exercise and activity, appropriate medical management of either hypertension or elevated blood glucose/diabetes, minimizing salt-intake and dietary modifications for both hypertension and diabetes including high-fiber, complex carbohydrates, and appropriate proteins and fats. Initial epidemiologic data appear to suggest that two of the more significant comorbidities associated with poor COVID-19 outcomes (especially when combined with age) are pre-existing hypertension and insulin resistance/Type II diabetes. First, these pre-existing conditions do not increase the risk of contracting COVID-19 but might increase the risk of more serious complications if the virus is contracted. Moreover, initial suggestions linking increased risk of infections in patients on current ACE inhibitors or angiotensin receptor blockers (ARBs) have not been validated. Regardless, innate genomic factors when combined with diet and lifestyle are strong predictors of both hypertension and insulin resistance. While it is unclear if or why hypertension and insulin resistance/Type II diabetes might be associated with poorer COVID-19 outcomes, what is clear is that these conditions can and do impair your optimal health. Anything that does the latter can diminish your immune system or exaggerate the pro-inflammatory effects of COVID-19.”

Antioxidation and Phase II Detox

Glutathionization is an important detoxification and anti-oxidation mechanism in human cells. The more optimal cells are at this process, the better they are at reducing the harmful effects of toxins and oxidants. Conversely, the less optimal cells are at glutathionization, the more at risk they are for being damaged by toxins and oxidants. A user's GST gene family may determine the efficiency of their cellular glutathionization.

Individuals with a deletion of both GSTT1 and GSTM1 and the AG or GG genotype of GSTP1 (about 5-7% of the general population) are significantly less optimal at cellular glutathionization—the utilization of glutathione and its precursor, NAC, to neutralize excess oxidants and toxins. These individuals are often much more sensitive to aerosolized toxins and inflammogens. As a simple barometer, they are often more sensitive to strong perfumes, air-fresheners, off-gassing from freshly painted rooms, fumes etc. They tend to be particularly sensitive to mold. Optimal vitamin C levels are an integral part of a cell's antioxidation capacity. Moreover, as efficient anti-oxidation is an important process in the reduction of the inflammatory cascade associated with infections, including that of SARS-Co-V2, individuals with poor cellular glutathionization capacity and suboptimal levels of vitamin C can be at a greater risk for more severe lower-respiratory inflammatory symptoms.

In some embodiments, the first relational model includes an association between the following genetic markers (genes and genotypes) and antioxidation and phase II detox:

GSTT1

CNV
0 copies: No enzyme activity with increased risk of oxidative and toxin damage
1 copy: Average enzyme activity average risk of oxidative and toxin damage
2 copies: Optimal enzyme activity reduced risk of oxidative and toxin damage

GSTM1

CNV
0 copies: No enzyme activity with increased risk of oxidative and toxin damage
1 copy: Average enzyme activity average risk of oxidative and toxin damage
2 copies: Optimal enzyme activity reduced risk of oxidative and toxin damage

GSTP1 RS1695
GG: Reduced enzyme activity with increased risk of oxidative and toxin damage
AG: Reduced enzyme activity increased risk of oxidative and toxin damage
AA: Optimal enzyme activity reduced risk of oxidative and toxin damage

SLC23A1

RS33972313
AA: significantly reduced absorption of vitamin C with increased risk of oxidative damage
AG: reduced absorption of vitamin C with increased risk of oxidative damage
GG: normal absorption of vitamin C with normal risk of oxidative damage

In an exemplary embodiment, the following phenotype questions may be included in the second relational model as probative of the antioxidation and phase II detox genotypes listed above from the first relational model:
1. Are you asthmatic or have tended to be asthmatic in the past?
2. Do you tend to get the common cold/flu more frequently than others?
3. When affected by a cold or flu, do you tend to develop difficulty breathing, congestion of the lungs, bronchitis, or pneumonia more easily (for example, if you recollect the last several flu seasons, did you develop any of these symptoms/conditions more easily than your peers/colleagues/family members)?
4. Have you ever been diagnosed with non-alcohol fatty liver disease?
5. Do you have a history of GI inflammatory presentations such as Crohn disease, Irritable Bowel Syndrome (IBD), Ulcerative Colitis?
6. Do you tend to be more sensitive to strong perfumes/artificial air-fresheners/freshly painted rooms etc.?
7. Do you tend to be sensitive to mold?
8. Do you tend to feel fatigued easily when exposed to the above inflammogens?
9. Do you tend to develop a sense of ‘brain fog’ when exposed to the above inflammogens?
10. Do you tend to be more easily fatigued or find it more difficult to recover from intense episodes of exercise?
11. Do you tend to crave sour or citrus fruits?
12. Have you ever found that increased vitamin C intake reduces the severity or duration of the common cold of flu?
13. Have you ever taken oral NAC or received glutathione IVs?
14. Do you feel better when you take oral NAC or received glutathione IVs?
15. Have you ever noticed that you take longer to recover from anesthetics?
16. Do you feel particularly horrible/toxified/fatigued after even moderate alcohol consumption?

In an exemplary embodiment, the predetermined set of rules for the third relational model may include the following rules associated with the antioxidation and phase II detox phenotype questions from the second relational model:

1. If at least one of questions 1-5 above has a yes/I don't know response, trigger an intervention recommendation for antioxidation and phase II detox; and

2. If at least four of questions 6-16 above have a yes/I don't know response, trigger the intervention recommendation for antioxidation and phase II detox.

The intervention recommendation for antioxidation and phase II detox may include a disclaimer, an action, and/or textual information. In an exemplary embodiment, the disclaimer for the vitamin D intervention may include the statement “Without appropriate genomic confirmation, we can only approximate your relative risk for genetic traits that impact your immune status and COVID-19 vulnerability through Anti-Oxidation and Phase 2 Detox.” In some embodiments, the disclaimer may additionally include the statement, “If you are on anti-depressants or other psychoactive medications, consult with your physician before adding NAC to your treatments.” For example, the additional statement above may be included in the disclaimer in response to a yes/I don't know response to one or more of questions 13 or 14 above. The action may include taking a dosage of one or more of: Vitamin C (maximum 2 gm/day); Selenium (as Selenium L. Methionine maximum 75 mg/day); R(+) Alpha-Lipoic Acid (maximum 75 mg/day); N-Acetyl Cysteine (maximum 600 mg/day); or Dietary Antioxidants (Flavonoids). In some embodiments, the action may be selected from amongst a set of actions, such as the set above. In some embodiments, the selection may be based on the answers to the phenotype questions above. For example, a positive answer to question 12 above may weigh selection of the action toward a dosage of Vitamin C. The textual information may include the statement “Interventions of potential benefit to you include appropriate intake of Vitamin C (Maximum 2 gm/day), Selenium (as Selenium L. Methionine maximum 75 mg/day) and R(+) Alpha-Lipoic Acid (maximum 75 mg/day), oral supplementation with N-Acetyl Cysteine (maximum 600 mg/day) and a diet rich in anti-oxidants, including dietary Flavonoids).” In some embodiments, the textual information may include a further statement with additional information, such as “Glutathionization is an important detoxification and anti-oxidation mechanism in your cells. The more optimal your cells are at this process, the better they are at reducing the harmful effects of toxins and oxidants. Conversely, the less optimal your cells are at glutathionization, the more at risk they are for being damaged by toxins and oxidants. Your GST gene family determines the efficiency of your cellular glutathionization. Individuals with a deletion of both GSTT1 and GSTM1 and the AG or GG genotype of GSTP1 (about 5-7% of the general population) are significantly less optimal at cellular glutathionization—the utilization of glutathione and its precursor, NAC, to neutralize excess oxidants and toxins. These individuals are often much more sensitive to aerosolized toxins and inflammogens. As a simple barometer they are often more sensitive to strong perfumes, air-fresheners, off-gassing from freshly painted rooms, fumes etc. They tend to be particularly sensitive to mold. Optimal vitamin C levels are an integral part of your cell's antioxidation capacity. Moreover, as efficient anti-oxidation is an important process in the reduction of the inflammatory cascade associated with infections, including that of SARS-Co-V2, individuals with poor cellular glutathionization capacity and suboptimal levels of vitamin C can be at a greater risk for more severe lower-respiratory inflammatory symptoms.”

Mitochondrial Redox Reaction

A symptom commonly associated with viral infections is the increase in cellular reactive oxygen species (ROS). Some degree of cellular ROS may actually be beneficial for cellular function. However, during viral infections, the antioxidant defense system of the cell can be overwhelmed, and cells can be exposed to excess ROS. Maintaining a healthy balance of cellular ROS is referred to as redox homeostasis. The genes, superoxide dismutase 2 (SOD2) and glutathione peroxidase (GPX) play important roles in maintaining redox homeostasis. Interestingly, tipping cellular redox homeostasis in favor of excess ROS can enhance viral replication, leading to what is known as a viral infection loop—the infection increases ROS and the increased ROS favors further viral replication.

Another parameter for viral replication and pathogenicity is the availability of host-cell micronutrients. During a viral infection, the nutrients and inner machinery of the cell are essentially hijacked for the purpose of viral replication. A key micronutrient implicated in the pathogenicity of viral infections is selenium, which is an essential component of a family of enzyme/proteins known as selenoproteins. GPX is an example of a selenoprotein. Genomic suboptimalities in both SOD2 and GPX, coupled with micronutrient insufficiencies of selenium and other important micronutrients and antioxidants, can create a cellular environment that favors viral replication.

In some embodiments, the first relational model includes an association between the following genetic markers (genes and genotypes) and the mitochondrial redox reaction:

SOD2

RS4880
CC: predisposed to optimal enzyme activity with increased conversion of O₃— to H₂O₂
CT: predisposed to moderate enzyme activity with moderate conversion of O₃— to H₂O₂
TT: predisposed to suboptimal enzyme activity with suboptimal conversion of O₃— to H₂O₂

GPX

RS1050450
CC: predisposed to optimal enzyme activity with increased conversion of H₂O₂ to
H₂O
CT: predisposed to moderate enzyme activity with moderate conversion of H₂O₂ to H₂O TT: predisposed to suboptimal enzyme activity with suboptimal conversion of H₂O₂ to H₂O

In an exemplary embodiment, the following phenotype questions may be included in the second relational model as probative of the mitochondrial redox reaction genotypes listed above from the first relational model:
1. Do you tend to feel fatigued easily when exposed to inflammogens?
2. Do you tend to develop a sense of ‘brain fog’ when exposed to inflammogens?
3. Do you have a history of GI inflammatory presentations such as Crohn disease, Irritable Bowel Syndrome (IBD), Ulcerative Colitis?
4. Do you tend to be more easily fatigued or find it more difficult to recover from intense episodes of exercise?
5. Do symptoms of a cold or flu infection tend to come on very quickly and strongly (simply stated, when you get a cold or flu do you go from feeling well to feeling ill very quickly?)
6. Have you gone grey prematurely/early?
7. Do you noticeably feel more energized or mentally alert when you take an antioxidant?
8. Do antioxidants help you recover from your exercise regiments?
9. Do antioxidants help you feel better or recover sooner from the common cold or flu?

In an exemplary embodiment, the predetermined set of rules for the third relational model may include the following rule associated with the mitochondrial redox reaction phenotype questions from the second relational model:

1. If at least four of questions 1-9 above has a yes/I don't know response, trigger an intervention recommendation for mitochondrial redox reaction.

The intervention recommendation for mitochondrial redox reaction may include a disclaimer, an action, and/or textual information. In an exemplary embodiment, the disclaimer for the vitamin D intervention may include the statement “Without appropriate genomic confirmation, we can only approximate your relative risk for genetic traits that impact your immune status and COVID-19 vulnerability through Mitochondrial Redox.” The action may include taking a dosage of one or more of Vitamin E (example Tocotrienol, maximum 100 mg/day); Acetyl-L-Carnitine (maximum 315 mg/day); Co-enzyme Q10 (maximum 200 mg/day); Vitamin D3 (maximum 2000 IU/day); Manganese (maximum 2.5 mg/day); or Selenium (as Selenium L. Methionine maximum 75 mg/day). The textual information may include the statement “Interventions of potential benefit to you include dietary support with micro-nutrients, including Vitamin E (example Tocotrienol, maximum 100 mg/day), Acetyl-L-Carnitine (maximum 315 mg/day), Co-enzyme Q10 (maximum 200 mg/day), Vitamin D3 (maximum 2000 IU/day) Manganese (maximum 2.5 mg/day) and Selenium (as Selenium L. Methionine maximum 75 mg/day).” In some embodiments, the textual information may include a further statement with additional information, such as “A hallmark of viral infections is the increase in cellular reactive oxygen species (ROS). Some degree of cellular ROS is actually beneficial for cellular function. However, during viral infections, the antioxidant defense system of the cell can be overwhelmed, and cells can be exposed to excess ROS. Maintaining a healthy balance of cellular ROS is referred to as redox homeostasis. The genes superoxide dismutase 2 (SOD2) and glutathione peroxidase (GPX) play important roles in maintaining redox homeostasis. Interestingly, tipping cellular redox homeostasis in favor of excess ROS can enhance viral replication, leading to what is known as a viral infection loop—the infection increases ROS and the increased ROS favors further viral replication. Another important parameter for viral replication and pathogenicity is the availability of host-cell micronutrients. During a viral infection, the nutrients and inner machinery of the cell are essentially hijacked for the purpose viral replication. A key micronutrient implicated in the pathogenicity of viral infections is selenium, which is an essential component of a family of enzyme/proteins known as selenoproteins. GPX is an example of a selenoprotein. Genomic sub-optimalities in both SOD2 and GPX, coupled with micronutrient insufficiencies of selenium and other important micronutrients and antioxidants, can create a cellular environment that favors viral replication.”

Methylation and Anti-Inflammation

In the context of the COVID-19 outbreak, cytokine storms, lower respiratory distress, acute lung injury, and pneumonia may each be related to the most significant morbidity associated with the infection—acute inflammation. Methylation is one of the most important of cellular processes designed to counter inflammation. Various combinations of suboptimal genotypes for the genes involved in cellular methylation process can strongly predispose an individual to poor anti-inflammatory responses and, accordingly, to potentially more severe symptomologies.

In some embodiments, the first relational model includes an association between the following genetic markers (genes and genotypes) and methylation and/or anti-inflammation:

SHMT1

RS1979277
GG: predisposed to optimal enzyme activity with optimal cellular methylation
GA: predisposed to suboptimal enzyme activity with suboptimal cellular methylation
AA: predisposed to suboptimal enzyme activity with suboptimal cellular methylation

MTHFR

RS1801133
CC: predisposed to optimal enzyme activity with optimal cellular methylation
CT: predisposed to moderate enzyme activity with moderate cellular methylation
TT: predisposed to suboptimal enzyme activity with suboptimal cellular methylation

MTHFR

RS1801131
AA: predisposed to optimal enzyme activity with optimal cellular methylation
AC: predisposed to moderate enzyme activity with moderate cellular methylation
CC: predisposed to suboptimal enzyme activity with suboptimal cellular methylation

FUT2

RS601338
AA: predisposed to optimal enzyme activity with optimal B12 absorption
AG: predisposed to suboptimal enzyme activity with suboptimal B12 absorption
GG: predisposed to suboptimal enzyme activity with suboptimal B12 absorption

TCN2

RS1801198
CC: predisposed to optimal enzyme activity with optimal B12 absorption
CG: predisposed to suboptimal enzyme activity with suboptimal B12 absorption
GG: predisposed to suboptimal enzyme activity with suboptimal B12 absorption

MTRR

RS1801394
AA: predisposed to optimal enzyme activity with optimal B12 methylation
AG: predisposed to suboptimal enzyme activity with suboptimal B12 methylation
GG: predisposed to suboptimal enzyme activity with suboptimal B12 methylation

MTR

RS1805087
AA: predisposed to optimal enzyme activity with optimal homocysteine methylation
AG: predisposed to suboptimal enzyme activity with suboptimal homocysteine methylation
GG: predisposed to suboptimal enzyme activity with suboptimal homocysteine methylation

COMT

RS4680
GG: predisposed to fast enzyme activity with fast substrate methylation
GA: predisposed moderate enzyme activity with moderate substrate methylation
AA: predisposed slow enzyme activity with slow substrate methylation

In an exemplary embodiment, the following phenotype questions may be included in the second relational model as probative of the methylation and/or anti-inflammation genotypes listed above from the first relational model:
1. Have you ever been tested for and found to have a low or suboptimal serum vitamin B12 level?
2. Have you ever had a high blood homocysteine test result?
3. Do you tend to develop symptoms of myalgia (muscle aches, often described as deep muscle aches) when you contract the common cold or flu?
4. Do you have a history of GI inflammatory presentations such as Crohn disease, Irritable Bowel Syndrome (IBD), Ulcerative Colitis?
5. Do you have a history of fibromyalgia and/or chronic fatigue syndrome?
6. Do you suffer from, or have ever suffered from a cardiovascular disease (including stroke, ischemic attacks, heart attacks)?
7. Do you suffer from, or have ever suffered from high cholesterol?
8. Are you on, or have ever been on, a statin or other lipid-lowering medications?
9. Are you a strict vegetarian or vegan?
10. Do you feel more energized when you take a vitamin B12 or B-complex supplement (including a vitamin B12 injection)?
11. Do you tend to get the common cold/flu more frequently than others?
12. When affected by a cold or flu, do you tend to develop difficulty breathing, congestion of the lungs, bronchitis, or pneumonia more easily (for example, if you recollect the last several flu seasons, did you develop any of these symptoms/conditions more easily than your peers/colleagues/family members)?
13. Do you tend to feel fatigued easily when you contract the common cold or flu?
14. Do you tend to develop a sense of ‘brain fog’ when you contract the common cold or flu?
15. Do you find it more difficult to recover from intense episodes of exercise?
16. Do muscle/ligament/tendon injuries tend to take long to heal?
17. Have you ever received a B12 injection and/or an IV with vitamin B's and subsequently felt nauseous, developed a headache or felt temporarily very fatigued?
18. Does the inflammation from insect bites tend to be more exaggerated or tend to stay longer?
19. Do food allergies tend to trigger myalgias or achy joints?
20. Do food allergies tend to trigger swollen gums and or inflamed nail beds/cuticles?
21. Do vitamin B supplements of any kind make you feel better when you have a cold or common flu?

In an exemplary embodiment, the predetermined set of rules for the third relational model may include the following rules associated with the methylation and/or anti-inflammation phenotype questions from the second relational model:

1. If at least one of questions 1-9 above has a yes/I don't know response, trigger an intervention recommendation for methylation and/or anti-inflammation; and

2. If at least three of questions 10-16 above have a yes/I don't know response, trigger the intervention recommendation for methylation and/or anti-inflammation.

3. If at least one of questions 17-21 above have a yes/I don't know response, trigger the intervention recommendation for methylation and/or anti-inflammation.

The intervention recommendation for methylation and/or anti-inflammation may include a disclaimer, an action, and/or textual information. In an exemplary embodiment, the disclaimer for the methylation and/or anti-inflammation intervention may include the statement “Without appropriate genomic confirmation, we can only approximate your relative risk for genetic traits that impact your immune status and COVID-19 vulnerability through Methylation and Anti-Inflammation Concerns.” The action may include taking a dosage of one or more of Thiamine (maximum 50 mg/day); Riboflavin (maximum 50 mg/day); Niacin (maximum 50 mg/day); Pantothenic Acid (maximum 90 mg/day); Folinic Acid (maximum 5 mg/day); Pyrodixine HCL (maximum 90 mg/day); Biotin (maximum 200 micrograms/day); Inositol (maximum 50 mg/day); Adenosyl B12 (maximum 3000 micrograms/day); Vitamin D3 (maximum 2000 IU/day); Trimethylglycine (maximum 200 mg/day); Black Cumin (N. sativa as oil maximum 3 tsp/day); Thunder-God Vine (T. wilfordii maximum 350 mg/day); or Turmeric (C. longa maximum 500 mg/day). In some embodiments, different actions are associated with different rules. For example, in some embodiments, Turmeric (C. longa maximum 500 mg/day) is only associated with a trigger resulting from rule 2 above, and Folinic Acid (maximum 5 mg/day) and Adenosyl B12 (maximum 3000 micrograms/day) are only associated with a trigger resulting from rule 3. The textual information may include the statement “Interventions of potential benefit to you include optimal B Vitamin supplementation (Thiamine maximum 50 mg/day, Riboflavin maximum 50 mg/day, Niacin maximum 50 mg/day, Pantothenic Acid maximum 90 mg/day, Folate maximum 1 mg/day, Pyridoxine HCL maximum 90 mg/day, Biotin maximum 200 micrograms/day, Inositol maximum 50 mg/day), Adenosyl B12 (maximum 3000 micrograms/day), Vitamin D3 (maximum 2000 IU/day) and Trimethylglycine (maximum 200 mg/day).” In some embodiments, the textual information may include a further statement with additional information, such as “In the context of the COVID-19 outbreak, cytokine storms, lower respiratory distress, acute lung injury, and pneumonia are all being discussed and are all related to the most significant morbidity associated with this infection—acute inflammation. Methylation is one of the most important of your cellular processes designed to counter inflammation. Various combinations of suboptimal genotypes for the genes involved in your cellular methylation process can strongly predispose you to poor anti-inflammatory responses and, accordingly, to potentially more severe symptomologies. Currently, a great deal of attention has been focused on the potential impact of ‘immunomodulation’ on cytokine storms, particularly concerning the potential benefit of anti-malarial drugs like Chloroquine and Hydroxychloroquine, which have also been used for the treatment of auto-immune disorders like Rheumatoid arthritis. Both drugs are synthetic forms of Quinine, itself derived from Cinchona Bark, which is now only used to flavor the bar-mix soda called tonic water because of safety concerns. Safer known ‘complementary and alternative medicine-based’ anti-inflammatory plant-based therapies include Black Cumin (N. sativa maximum dose oil 3 tsp/day) and Turmeric (C. longa maximum 500 mg/day).”

Mood and Behavior

The COVID-19 pandemic, and the lifestyle upheavals that may be associated with it, may be a once in a generation phenomenon. Many within our societies may not have experienced the like of this occurrence before. How individuals react to the increasingly severe domino-like consequences of this pandemic is not trivial, and may have profound and/or lasting mental or sociological effects. By virtue of their innate genomic predispositions, huge segments of our societies are going through an odious, under-appreciated phenomenon and not even realize it—large scale psychological and emotional trauma.

Individuals with fast COMT genotypes, especially when paired with fast MAO genotypes (and exaggerated with genotypes that reduce DRD2 expression) may be at an increased risk of depression. This risk may be further heightened by poor BDNF production. This may be precipitated in the context of isolation, fear over one's livelihood, fear of illness for oneself and one's loved ones etc. Low vitamin D (as discussed above) may correlate strongly with low dopamine, and may also influence BDNF production. BDNF is one of the most important circadian rhythm genes and may be associated with human behavior. Low BDNF amplifies circular thought patterns related to emotional stress, creating a cyclical regurgitation of negative thoughts. Optimal BDNF may be associated with consistent and healthy daily circadian rhythms, access to sunlight, and exercise—the very lifestyle components that may suffer during pandemics such as the COVID-19 outbreak.

As opposed to individuals with the fast COMT and MAO related genotypes, those with the slow version of these genes may be at an increased risk of anxiety. Behaviorally, the phenotypes for fast versus slow versions of these genes can cross-over—increased anxiety over an environment/pandemic can influence a propensity toward depression. However, individuals with the slow versions of COMT and MAO may generally tend to overreact and be hypersensitive to news, and to perceived threats associated with the pandemic. Rather than be weighed by a sense of inertia (that might otherwise be observed in the previous genomic demographic), these are the individuals that may tend to take rash, socially negative actions such as flocking to the stores to panic buy and horde. In general, they may tend to over-react to any and all emotional cues. Their emotional latency may be strongly linked to an increased risk of elevated cortisol, with co-morbidities of hypertension—singularly the highest co-morbidity predictive of poor outcome with SARS-Co-v2 infection. This tendency to hyper-emotional responses may be exaggerated with the D allele of the ADRA2B gene, the S allele of the SLC6A4 gene, the T allele of the TPH2 gene, and the A allele of the BDNF gene. Particular care and consideration should be given to children with this profile. Displaced school regiments, observing mom and dad and elders reacting negatively, and of course, their own potential over-consumption of social media and the negative messages being promulgated therein, are significant factors in exacerbating latent tendencies of anxiety and mood disorders in this group of children.

In some embodiments, the first relational model includes an association between the following genetic markers (genes and genotypes) and mood and behavior issues:

COMT

RS4680
GG: predisposed to fast enzyme activity with fast neurochemical neutralization
GA: predisposed to moderate enzyme activity with moderate neurochemical neutralization
AA: predisposed to slow enzyme activity with slow neurochemical neutralization

MAO

RS6323
GG: predisposed to fast enzyme activity with fast neurochemical neutralization
GT: predisposed to moderate enzyme activity with moderate neurochemical neutralization
TT: predisposed to slow enzyme activity with slow neurochemical neutralization

DRD2

RS1800497
GG: predisposed to higher dopamine receptor expression and intense response to dopamine
GA: predisposed to moderate dopamine receptor expression and moderate response to dopamine
AA: predisposed to low dopamine receptor expression and reduced response to dopamine

ADRA2B

INDEL
DD: predisposed to prolonged noradrenalin receptor activation and intense response to noradrenalin
ID: predisposed to prolonged noradrenalin receptor activation and intense response to noradrenalin
II: predisposed to shortened noradrenalin receptor activation and reduced response to noradrenalin

SLC6A4

INDEL
SS: predisposed to reduced serotonin secretion and reduced response to serotonin
LS: predisposed to reduced serotonin secretion and reduced response to serotonin
LL: predisposed to optimal serotonin secretion and optimal response to serotonin

TPH2

RS4570625
TT: predisposed to fast enzyme activity with fast serotonin neutralization
GT: predisposed to moderate enzyme activity with moderate serotonin neutralization
GG: predisposed to slow enzyme activity with slow serotonin neutralization

BDNF

RS6265
GG: predisposed to higher BDNF expression and optimal neural plasticity
GA: predisposed to lower BDNF expression and lower neural plasticity
AA: predisposed to lower BDNF expression and lower neural plasticity

In an exemplary embodiment, the following phenotype questions may be included in the second relational model as probative of the mood and behavior genotypes listed above from the first relational model:
1. Do you suffer from, or ever suffered from, depression?
2. Are you currently on, or have ever been on, anti-depressives?
3. Do you suffer from, or ever suffered from, anxiety?
4. Are you currently on, or have ever been on, anti-anxiety medications?
5. Do you suffer from, or ever suffered from, any other form of mood/behavioral disorder (ADD, ADHD, OCD, Bi-polar, etc.)?
6. Are you currently on, or have ever been on, medications for any mood/behavioral disorders in point 5 above?
7. Do you suffer from, or have ever had, any addictions or substance abuse tendencies?
8. Have you ever been diagnosed with PTSD?
9. Do you tend to be a ‘cup half empty’ person?
10. Do you tend to become easily saddened or feel a sense of despondence?
11. Do you tend to become easily anxious or feel a sense of irrational fear?
12. When a problem arises, do you tend to think of the worse outcomes that might occur?
13. If asked, would friends or relatives say that you tend to over-react to emotional stimuli (whether positive or negative)?
14. If asked, would friends or relatives say that you tend to under-react to emotional stimuli (whether positive or negative)?
15. When faced with a negative emotional event, do you tend to fixate on the event (after it has passed) or continually replay its circumstances?
16. Do you tend to feel easily deflated without positive/pleasurable stimuli?
17. Do loved ones or friends/colleagues consider you apathetic, that is, you tend not to be able to sense the emotions (or care to sense them) of others and respond appropriately?
18. Do you consider yourself a taker (do you tend to see to your needs and wants before you think of the needs and wants of others)?
19. Do you feel trapped in long-term relationships?
20. Do you consider yourself a giver (do you tend to see to the needs of others before that of yourself)?
21. When a problem arises, do you tend to quickly get past thinking of negative outcomes and focus more easily on a possible solution?
22. Do you tend to be a ‘cup half full’ person?
23. Can you easily put the feelings of others before yourself?
24. Do you feel happy and contented in a long-term relationship?
25. Do loved ones or friends/colleagues consider you empathetic, that is, are you able to sense the emotions of others and respond appropriately?
26. Do you tend to feel easily elated with positive/pleasurable stimuli?

In an exemplary embodiment, the predetermined set of rules for the third relational model may include the following rules associated with the mood and behavior phenotype questions from the second relational model:

1. If at least one of questions 1-8 above has a yes/I don't know response, trigger an intervention recommendation for mood and behavior;

2. If at least three of questions 9-19 above have a yes/I don't know response, trigger the intervention recommendation for mood and behavior; and

3. If at least three of questions 20-26 above have a yes/I don't know response, trigger the intervention recommendation for mood and behavior.

The intervention recommendation for mood and behavior may include a disclaimer, an action, and/or textual information. In an exemplary embodiment, the disclaimer for the mood and behavior intervention may be selected based on the rule triggering the recommendation. For example, a disclaimer for a trigger from the first rule may include the statement “Without appropriate genomic confirmation, we can only approximate your relative risk for genetic traits that impact your immune status and COVID-19 vulnerability through Mood and Behavior Concerns. However, you have provided a positive response to x/y questions of concern. If you are concerned that the current pandemic will trigger or worsen your anxiety and/or depression, we strongly advise that you speak to your healthcare provider to ensure you are receiving appropriate support.” A disclaimer for a trigger from the second rule may include the statement “Without appropriate genomic confirmation, we can only approximate your relative risk for genetic traits that impact your immune status and COVID-19 vulnerability through Mood and Behavior Concerns. However, you have provided a positive response to x/y questions of concern. The societal, monetary, and emotional impacts of the COVID-19 pandemic are new to many and may exaggerate your risk of depression and/or anxiety. If at any time you or your loved ones observe that your mental and/or emotional health is less than optimal, we strongly encourage you to speak to a qualified healthcare provider.” A disclaimer for a trigger from the third rule may include the statement “Without appropriate genomic confirmation, we can only approximate your relative risk for genetic traits that impact your immune status and COVID-19 vulnerability through Mood and Behavior Concerns. However, you have provided a negative response to x/y questions of concern. The societal, monetaryt, and emotional impacts of the COVID-19 pandemic are new to many and may exaggerate your risk of depression and/or anxiety. If at any time you or your loved ones observe that your mental and/or emotional health is less than optimal, we strongly encourage you to speak to a qualified healthcare provider.” In some embodiments, an additional disclaimer may be added in response to an indication that the user is on an anti-depressant, such as the statement “If you are on anti-depressants or other psychoactive medications, consult with your physician before adding NAC to your treatments.”

The action may include taking a dosage of one or more of Vitamin D3 (maximum 2000 IU/day); S adenylmethionine (maximum 400 mg/day); N-acetyl Cysteine (maximum 600 mg/day). The action may be selected based on the rule causing the trigger. For example, the action may only include S adenylmethionine (maximum 400 mg/day) for a rule 1 trigger. The textual information may include the statement “Interventions of potential benefit to you include Vitamin D3 supplementation (maximum 2000 IU/day), maximizing your safe-sun exposure, particularly in the winter months, oral N-acetyl Cysteine supplementation (maximum 600 mg/day) for risk of anxiety/OCD and supplementary S-adenylmethionine (maximum 400 mg/day) for risk of depression.” In some embodiments, the textual information may include a further statement with additional information, such as “This pandemic and the lifestyle upheavals that it has caused, and will continue to cause, is a once in a generation phenomenon. Many within our societies have not experienced the like of this before. How we individually react to the increasingly severe domino-like consequences of this pandemic is not trivial at all. By virtue of their innate genomic predispositions, huge segments of our societies are going through an odious, under-appreciated phenomenon without even realizing it—large scale psychological and emotional trauma. Individuals with fast COMT genotypes, especially when paired with fast MAO genotypes (and exaggerated with genotypes that reduce DRD2 expression) are at an increased risk of depression. This risk is further heightened by poor BDNF production. This is precipitated in the current context of isolation, fear over one's livelihood, fear of illness for oneself and one's loved ones etc. Low vitamin D (see vitamin D symptom discussion above) correlates strongly with low dopamine and also influences BDNF production. BDNF is one of the most important circadian rhythm genes and also one of the most studied genes in human behavior. Low BDNF amplifies circular thought patterns related to emotional stress, creating a classic hamster wheel mental regurgitation of negative thoughts—not the best foundation upon which to filter and handle the near constant and sensationalized news highlights and feeds. Optimal BDNF requires consistent and healthy daily circadian rhythms, access to sunlight, and exercise—the very lifestyle components that suffer during pandemics such as the COVID-19 outbreak. In difference to individuals with the fast COMT and MAO related genotypes, those with the slow version of these genes are at an increased risk of anxiety. Behaviorally, the phenotypes for fast versus slow versions of these genes can cross-over—increased anxiety over the current environment/pandemic can obviously influence a propensity toward depression. However, individuals with the slow versions of COMT and MAO will generally tend to overreact and be hypersensitive to the news, and to perceived threats associated with this pandemic. Rather than be weighed by a sense of inertia (that might otherwise be observed in the previous genomic demographic), these are the individuals that will tend to flock to the stores and panic buy and horde. In general, they will tend to over-react to any and all emotional cues. Their emotional latency is strongly linked to an increased risk of elevated cortisol, with co-morbidities of hypertension—singularly the highest co-morbidity predictive of poor outcome with SARS-Co-v2 infection. This tendency to hyper-emotional responses is exaggerated with the D allele of the ADRA2B gene, the S allele of the SLC6A4 gene, the T allele of the TPH2 gene, and the A allele of the BDNF gene. Particular care and consideration should be given to children with this profile. Displaced school regiments, observing mom and dad and elders reacting negatively, and of course, their own potential over-consumption of social media and the negative messages being promulgated therein, are significant factors in exacerbating latent tendencies of anxiety and mood disorders in this group of children.”

In some embodiments, the assessment tool may determine a severity risk assessment for a particular symptom based on, for example, one or more of a quantity of phenotype questions with a positive response associated with that symptom, a number of rules for that symptom that were triggered, a number of interventions recommended to the user, or the like. In some embodiments, the severity risk assessment for a symptom may also be based on other information received or retrieved by the assessment tool, such as the preliminary question answers, etc. In some embodiments, a particular recommendation action may only be provided to the user in response to that action being associated with a predetermined number of positive phenotype questions, triggered rules, or the like. Any acceptable metric for a severity risk assessment may be used such as, for example, a selection from mild, moderate, and high, or the like, a selection of a number along a numerical range, a selection of a color along a gradient, or the like.

It should be understood that the genotypes, phenotype questions, rules, and interventions discussed above are exemplary, and that other genotypes, phenotypes, rules, and interventions may be associated with COVID-19 in particular, and with other illnesses in general, as well as symptoms of those illnesses and associated genotypes, phenotypes, and interventions, etc.

As discussed above, the relationship between symptom or illness severity and genotype or phenotype may not be fully understood. Further, illnesses and/or the way an illness is expressed may change due to various factors, such as modification or evolution of the disease, environmental factors, or the like. In some embodiments, the assessment tool may be configured to retrieve and/or receive data associated with relationships between an illness, a symptom, a genotype, and/or a phenotype, and generate, tune, update, or adjust at least one relational model based on such data.

In an example, the assessment tool may track symptom duration and severity for persons who have contracted the illness. In some embodiments, the assessment tool may prompt users to indicate whether they have contracted the illness. In some embodiments, the assessment tool may prompt users who have contracted the illness to enter information associated with the severity and duration of their symptoms. In some embodiments, such a prompt may be periodic, may occur in response to the user accessing the assessment tool or logging in to a user account associated with the assessment tool, may occur at a predetermined time or according to a predetermined schedule, or the like. In some embodiments, the assessment tool may retrieve and/or receive data on symptom severity and duration from another source such as, for example, the provider system 110, or the like. In some embodiments, the assessment tool may generate, update, tune, or adjust at least one relational model based on the tracked data. In some embodiments, the assessment tool and/or the assessment system 120 or another system may anonymize the tracked data and or other data received and/or retrieved by the assessment tool.

For example, in some embodiments the tracked data may be used along with the first relational model between symptoms of a disease and genotypes associated with such systems. The assessment tool may prompt for, retrieve, and/or receive genetic information for the users providing the tracked symptom duration and severity data. The assessment tool may compare the received genetic information with the tracked data to identify genotypes that may be associated with more or less severe symptoms. In some embodiments, the assessment tool may employ one or more machine learning techniques to perform the comparison.

Any suitable machine learning technique may be used, such as linear regression, logistical regression, random forest, gradient boosted machine (GBM), and/or a deep neural network. A machine learning model may be initialized with no data, with random data, and/or with predetermined data such as the information for the first relational model discussed above. Supervised or unsupervised training may be employed. For example, unsupervised approaches may include K-means clustering. K-Nearest Neighbors may also be used, which may be supervised or unsupervised. Combinations of K-Nearest Neighbors and an unsupervised cluster technique may also be used. Any of N parameters associated with a symptom, portion of genetic code, or the like may correspond to a dimension in the model. As the machine learning system is trained, parameters that have been successful in identifying a relationship between a symptom severity and a genotype may form clusters in N-dimensional space. Simpler models may be deployed in parallel for speed. For example, rather than analyze all of a person's DNA, a small number of predetermined portions N—X may be fed to a simplified machine learning system to quickly determine if the predetermined portions are likely to be associated with a symptom. A machine learning relational model may be generated and trained, based on the genetic information as training data and on the tracked symptom data as ground truth, to identify portions of genetic code likely to be probative of a severity of a symptom. The machine learning relational model may form the first relational model, or may be used to replace, tune, or adjust the first relational model.

In another example, the data received and/or retrieved by the assessment tool may be used along with genetic information and the second relational model between genotypes and phenotype questions. The assessment tool may compare the received and/or retrieved data, such as answers to the phenotype questions, preliminary questions, lifestyle questions, demographic questions, medical history, etc., with the genetic data to determine factors that may be probative of one or more genotypes. In some embodiments, the assessment tool may determine that a particular factor is probative of a genotype, and add that factor to the second relational model and/or increase a weight of that factor in the second relational model. In some embodiments, the assessment tool may determine that a particular factor is less probative of a genotype, and may remove that factor from the second relational model and/or decrease a weight of that factor in the second relational model.

In some embodiments, the assessment tool may employ one or more machine learning techniques in developing the second relational model. A second machine learning model may be generated that is trained, based on the genetic information and the data received and/or retrieved by the assessment tool, to identify phenotype factors that may be probative of one or more genotypes. In some embodiments, the second machine learning model may be initialized with predetermined data from the second relational model, such as the data discussed above with regard to COVID-19.

In some embodiments, the assessment tool is configured to generate questions for factors added to the second relational model. In some embodiments, the assessment tool may employ a natural language processing algorithm to generate such questions. In some embodiments, the assessment tool is configured to transmit a request, e.g., to an entity associated with the assessment system 120, for entry of such questions.

In some embodiments, the assessment tool may utilize the tracked symptom data and the answers to the phenotype questions and/or the other data received and/or retrieved by the assessment tool along with the third relational model. In some embodiments, the assessment tool may compare the answers to the phenotype questions and/or the other data received and/or retrieved by the assessment tool to the symptom data in order to determine phenotype factors and/or combinations of phenotype factors that are probative of a particular severity of a symptom. The assessment tool may generate, adjust, add, or remove one or more rules for the third relational model based on the comparison. The assessment tool may employ one or more machine learning techniques in developing the third relational model.

In some embodiments, the assessment tool may prompt, receive, and/or retrieve information associated with interventions executed for users that have contracted the illness. The assessment tool may compare the tracked symptom data with the intervention data to determine an effectiveness on one or more intervention of reducing a severity of a symptom when performed prior to contracting the disease and/or reducing a severity or duration of a symptom when performed after the disease has been contracted.

In some embodiments, the assessment tool may employ a hybrid relational model that includes one or more aspects form more than one relational model. In some embodiments, the assessment tool may initially use one or more relational models generated with predetermined information, and may switch to a different relational model in response to satisfaction of a criteria. For example, in some embodiments, the assessment tool may employ predetermined relational models, such as the first, second, and third models associated with COVID-19 discussed above, and may switch over one or more of the models to a model generated, tuned, or updated using tracked symptom data in response to the assessment tool receiving and/or retrieved tracked symptom data from a predetermined threshold number of patients. Any suitable criteria may be used.

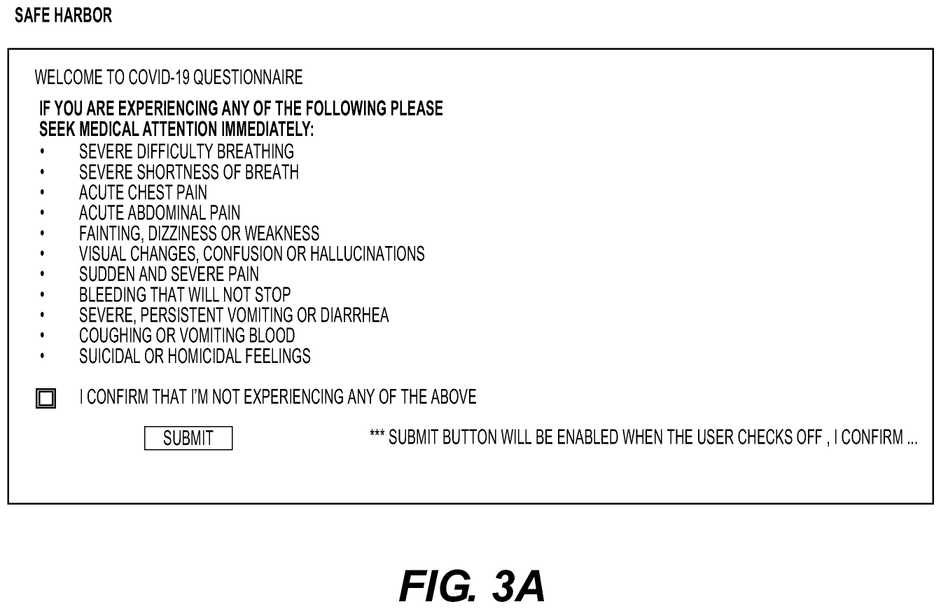

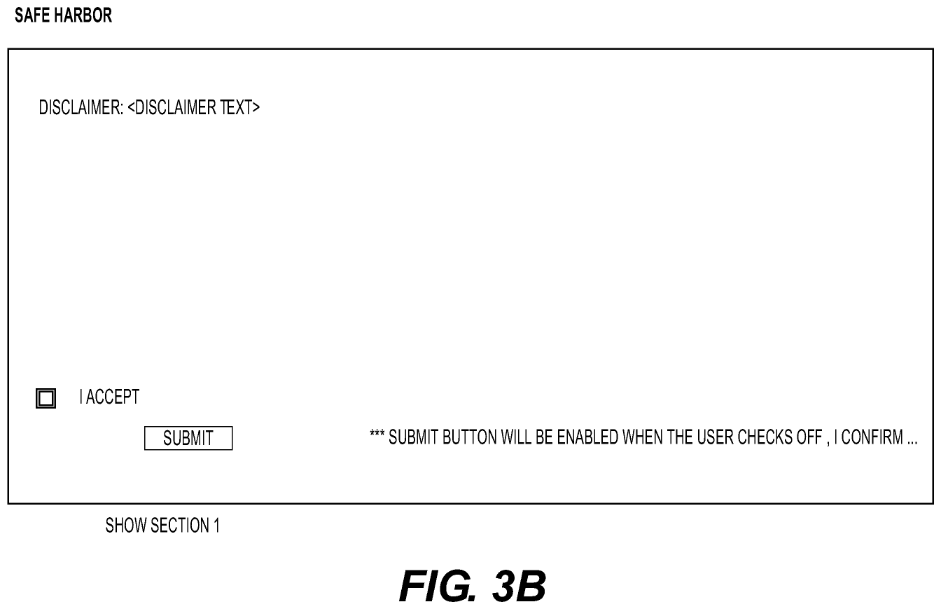

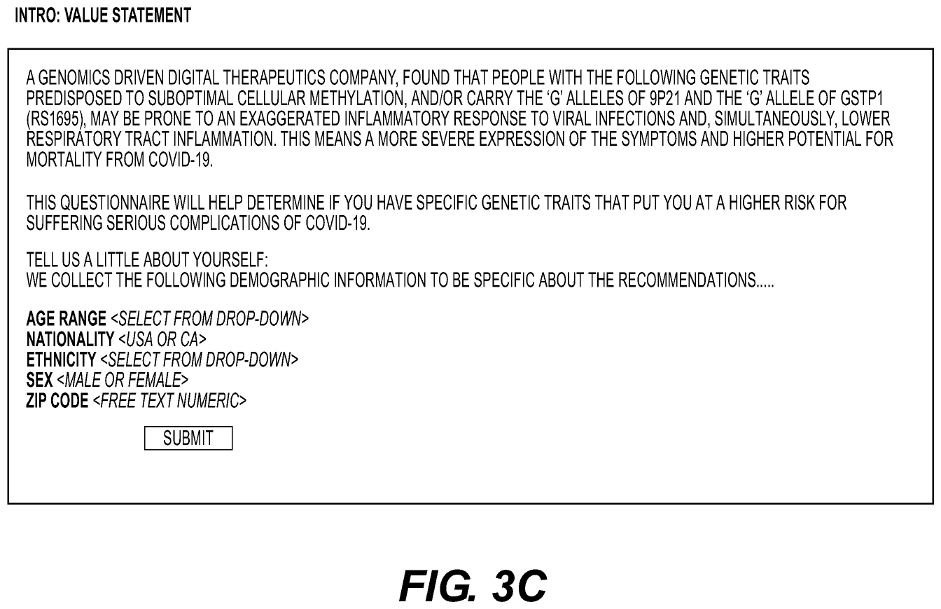

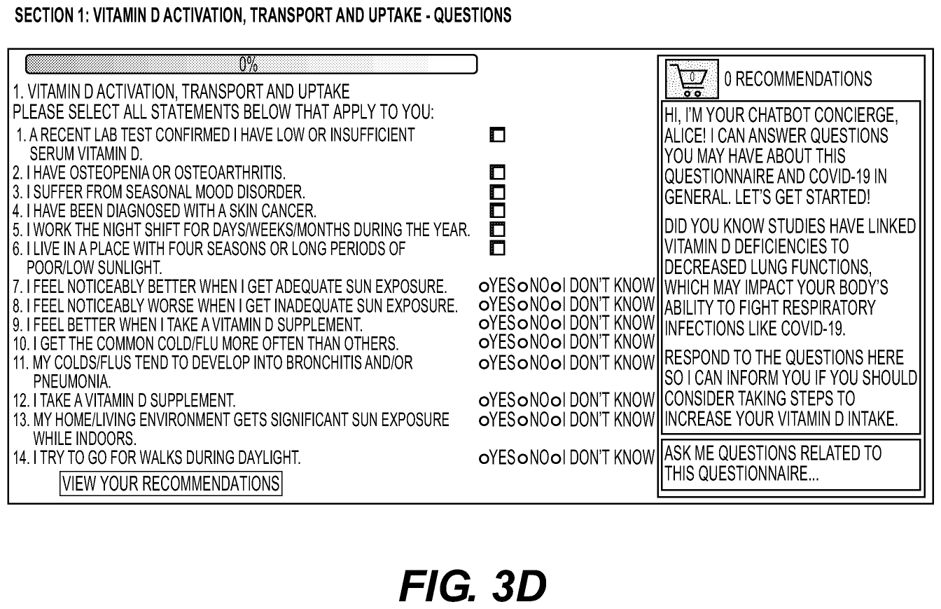

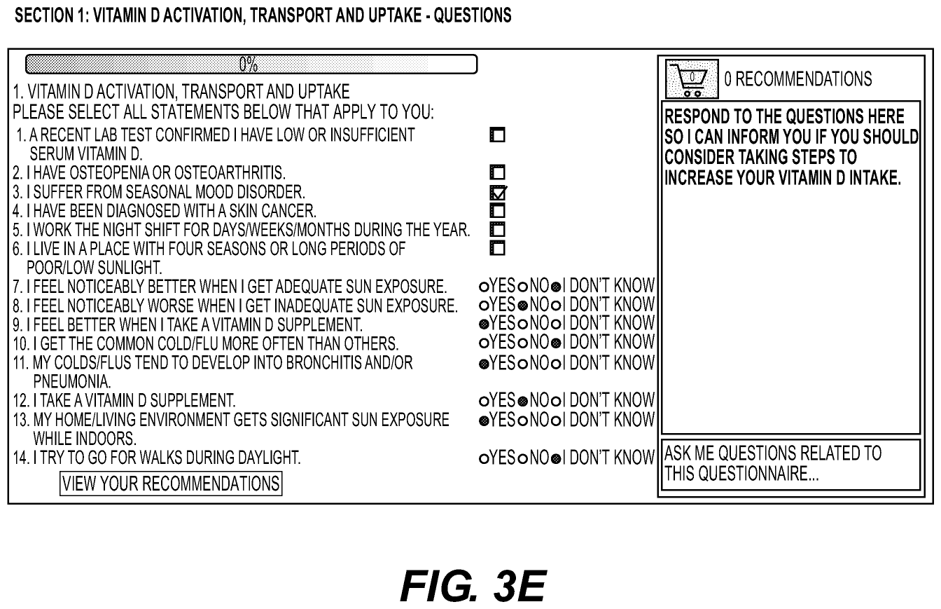

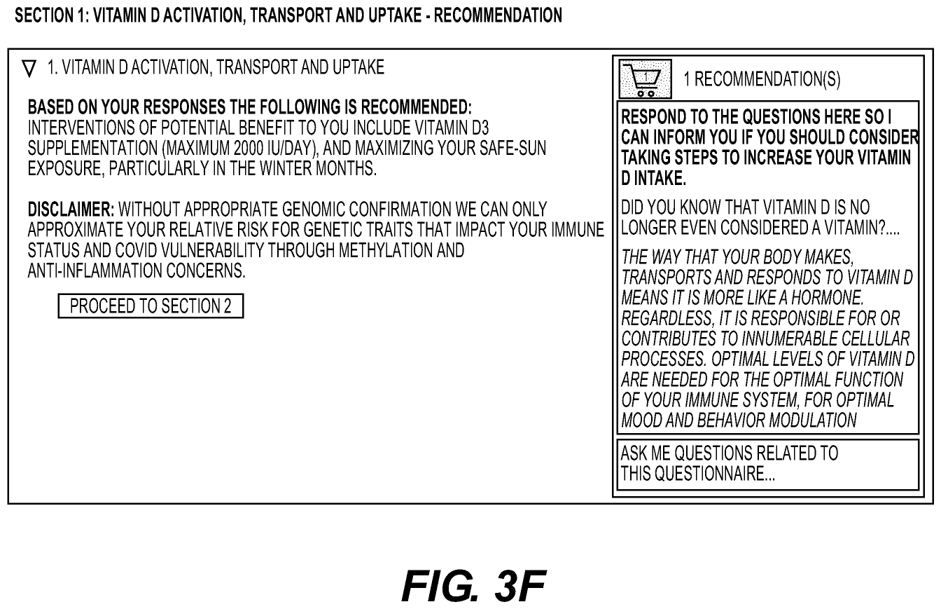

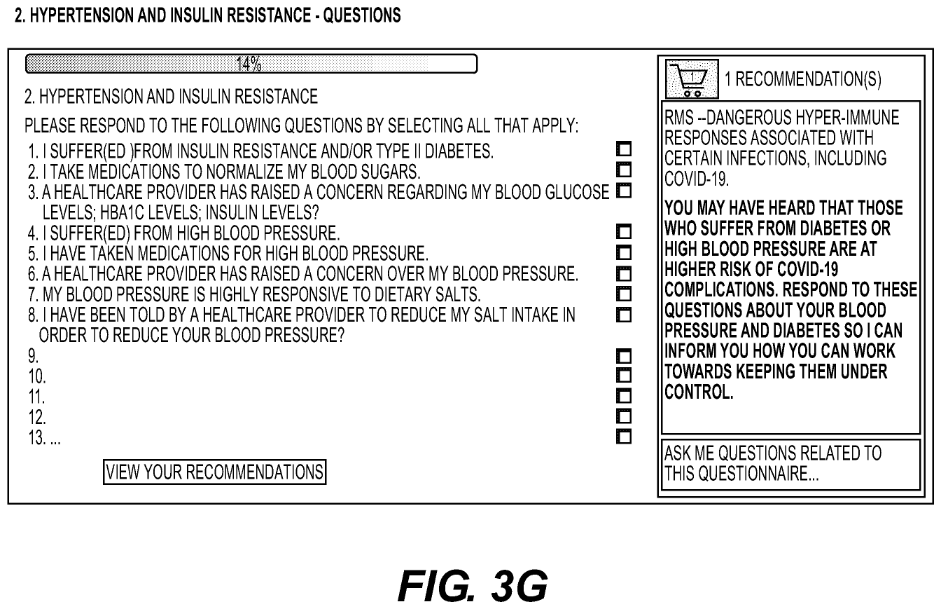

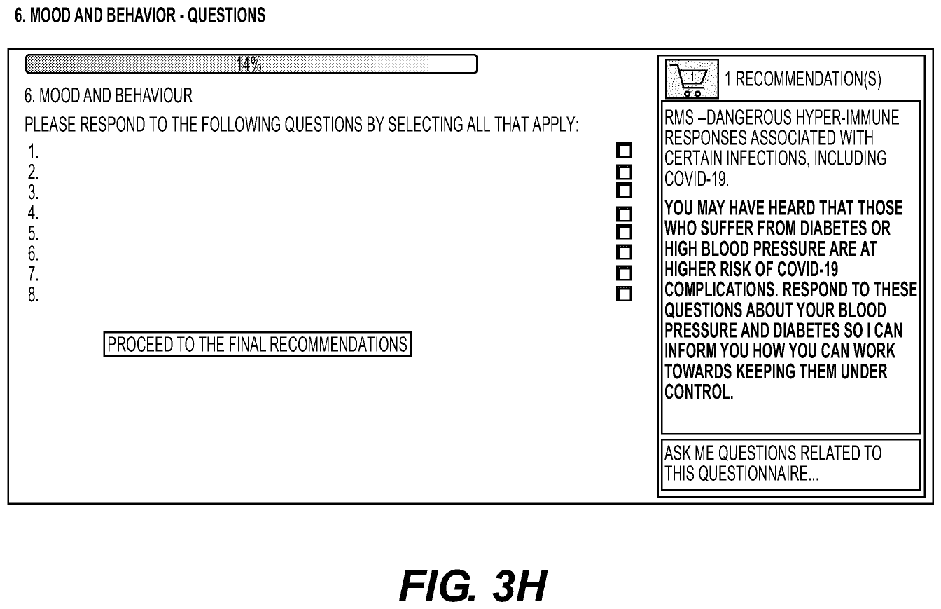

In some embodiments, the assessment tool may prompt for answers to all phenotype questions for all symptoms of a disease at once. In some embodiments, the assessment tool may separate the phenotype questions by symptom, by rule, or by any suitable criteria. In some embodiments, the assessment tool may provide a severity risk assessment and/or an intervention recommendation on a per-symptom basis as the phenotype questions for that symptom are answered. FIGS. 3A-3I depict an exemplary user interface for an assessment tool that implements one or more of the features discussed above.

In some embodiments, the assessment tool includes a chatbot, i.e., an artificial conversant that may be configured to receive text from the user via a user interface, and provide information related to the input text. In some embodiments, the chatbot may employ a natural language algorithm. In some embodiments, the chatbot may provide information based on the health assessment for the user, the other information retrieved and/or received by the assessment tool, one or more relational models, or the like. In some embodiments, one or more severity risk assessments for the user may be based on information received by or determined by the chatbot.

In some embodiments, the assessment tool may determine insights about a disease based on data for a plurality of users. For example, in some embodiments, the assessment tool may identify hot-spots for areas of people contracting the illness. In some embodiments, the assessment tool may identify areas with a relatively higher concentration of people likely to experience severe symptoms and/or to need intensive medical intervention. In some embodiments, the assessment tool may identify an impact on the illness by external factors such as environment, climate, season, weather, or the like, such as rate of contraction, severity of symptoms, duration of symptoms, etc. In some embodiments, the assessment tool may identify and/or track an effectiveness of one or more interventions on a symptom. Such data may be beneficial for combating and/or minimizing negative effects of the illness.

In some embodiments, the assessment tool may provide data such as the above and or other data related to users, user health assessments, relational models, etc. to one or more other entities. For example, in some embodiments, hot spot data and/or symptom severity data may be transmitted to the provider system 110 to facilitate identification of medical equipment, staff, capacity, and care needs. Providing such information before a hot-spot has broken out may provide medical professionals with the time needed to more completely prepare. Health assessment and tracked symptom recommendation may be proved to enable care providers to view if, when, and/or how a user is complying with the recommendations. Such information may assist a care provider in providing care, an insurer in determining costs or approving care, or the like. In some embodiments, such data may be provided to a regulating entity in order to inform decision making and policy. In some embodiments, such data may be anonymized or encrypted. In some embodiments, the assessment tool may employ a blockchain technique.

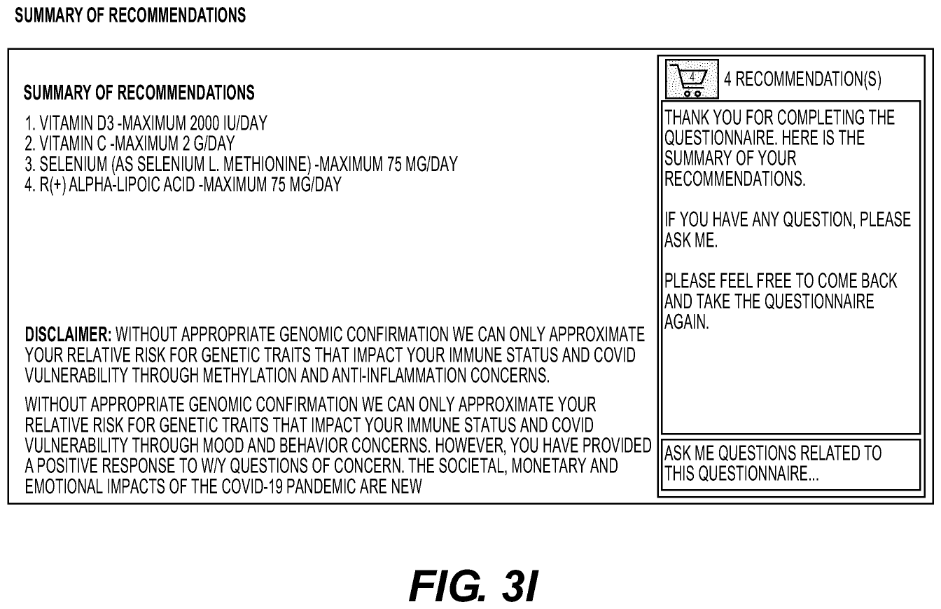

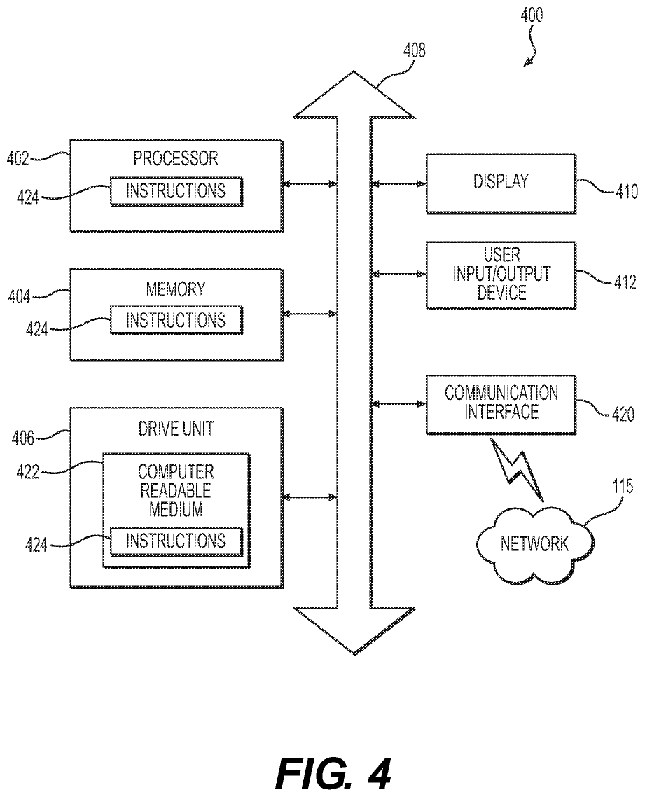

FIG. 4 is a simplified functional block diagram of a system 400 that may be configured as a device for executing the method of FIG. 2, according to exemplary embodiments of the present disclosure. FIG. 4 is a simplified functional block diagram of a computer that may be configured as the assessment system 120 according to exemplary embodiments of the present disclosure. Specifically, in one embodiment, any of the mobile devices, systems, servers, etc., discussed herein may be an assembly of hardware including, for example, a data communication interface 420 for packet data communication. The platform also may include a central processing unit (“CPU”) 402, in the form of one or more processors, for executing program instructions. The platform may include an internal communication bus 408, and a storage unit 406 (such as ROM, HDD, SDD, etc.) that may store data on a computer readable medium 422, although the system 400 may receive programming and data via network communications. The system 400 may also have a memory 404 (such as RAM) storing instructions 424 for executing techniques presented herein, although the instructions 424 may be stored temporarily or permanently within other modules of system 400 (e.g., processor 402 and/or computer readable medium 422). The system 400 also may include input and output ports 412 and/or a display 410 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the presently disclosed methods, devices, and systems are described with exemplary reference to transmitting data, it should be appreciated that the presently disclosed embodiments may be applicable to any environment, such as a desktop or laptop computer, an automobile entertainment system, a home entertainment system, etc. Also, the presently disclosed embodiments may be applicable to any type of Internet protocol.

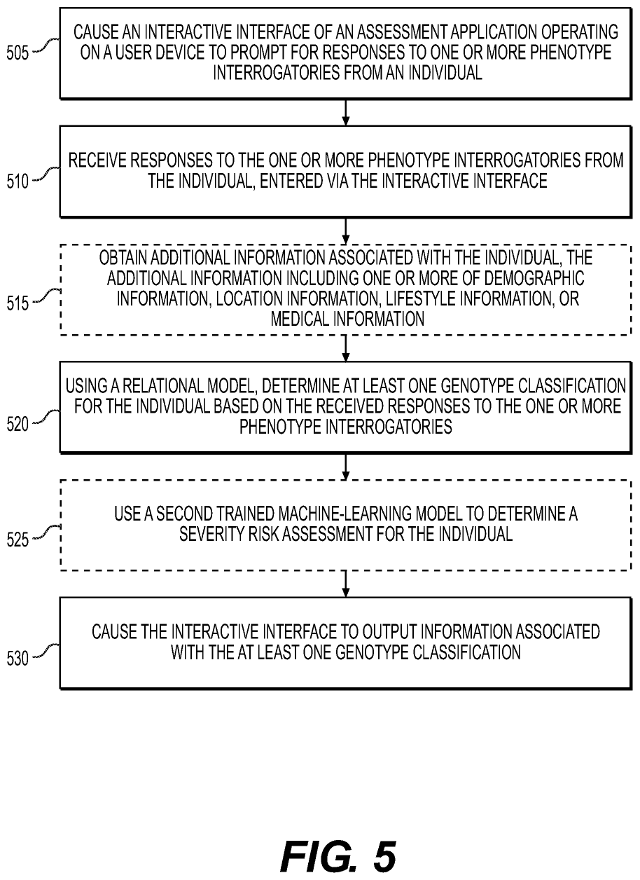

FIG. 5 depicts a flow diagram of an exemplary embodiment of a method for performing a genotype-based analysis of an individual. At step 505, the assessment system 120 may cause an interactive interface of an assessment application (e.g., such as the interface depicted in FIGS. 3A-3I) operating on the user device 105 to prompt for responses to one or more phenotype interrogatories from an individual, e.g., a user and/or patient. In some embodiments, the one or more phenotype interrogatories includes a plurality of phenotype interrogatories categorized into clusters, each cluster associated with a respective genotype.

At step 510, the assessment system 120 may receive, e.g., from the user device 105, responses to the one or more phenotype interrogatories from the individual, entered via the interactive interface.

Optionally, at step 515, the assessment system 120 may obtain additional information associated with the individual, the additional information including one or more of demographic information, location information, lifestyle information, or medical information. In various embodiment, the assessment system 120 may obtain the additional information via one or more of causing the interactive interface to prompt for response to one or more further interrogatories associated with the additional information; accessing a profile associated with the individual; or accessing a database including medical information associated with the individual.

At step 520, the assessment system 120 may, using a relational model, determine at least one genotype classification for the individual based on the received responses to the one or more phenotype interrogatories. In some embodiments, the at least one genotype classification includes at least one of a genotype for at least one gene of the individual, an indication that the individual has or is at risk for a symptom or illness, or a severity risk assessment of the symptom or illness for the individual. In some embodiments, the relational model is configured to further base the at least one genotype classification for the individual on the additional information. In some embodiments, the relational model is configured to assign respective weights to the responses. The respective weights may be, for example, indicative of one or more of a probative value of corresponding phenotype interrogatories to one or more genotypes, or a population prevalence of a phenotype or genotype associated with the corresponding genotype interrogatories.

In some embodiments, using the relational model includes using a first trained machine-learning model to determine one or more genotypes of the individual based on the received responses and learned associations of the first trained machine-learning model. The first trained machine-learning model may have been previously trained, based on (i) training phenotype interrogatories responses from one or more individuals and (ii) ground truth genotypes of the one or more individuals to learn associations between the training phenotype interrogatory responses and the ground truth genotypes. In some embodiments, the learned associations for the first trained machine-learning model include one or more different weights or groupings applied by the relational model to the responses.

In some embodiments, the at least one genotype classification includes the severity risk assessment. Optionally, at step 525, the assessment system 120 may use a second trained machine-learning model to determine the severity risk assessment for the individual, e.g., based on learned associations of the second trained machine-learning model and the one or more genotype classification for the individual, e.g., from the first trained machine-learning model. The second trained machine-learning model may have been previously trained, based on training genotypes for the one or more individuals and ground truth symptom or illness severity information for the one or more individuals to learn associations between the training genotypes and the ground truth severity information.

At step 530, the assessment system 120 may cause the interactive interface to output information associated with the at least one genotype classification. In various embodiments, the information associated with the at least one genotype classification includes one or more of: a list of one or more symptoms or illnesses for which the individual is at risk; or an intervention recommendation associated with the one or more symptoms or illnesses.

In some embodiments, the information associated with the at least one genotype classification may be determined via a further machine-learning model. For example, in embodiments in which the information associated with the at least one genotype classification includes the intervention recommendation, the assessment system 120 may use a third trained machine-learning model to determine the intervention recommendation based on learned associations of the third trained machine-learning model and the one or more genotype classification for the individual. The third trained machine-learning model may have been previously trained, based on training intervention use and results information for the one or more individuals and the ground truth genotypes for the one or more individuals to learn associations between the training use and results information and the ground truth genotypes.

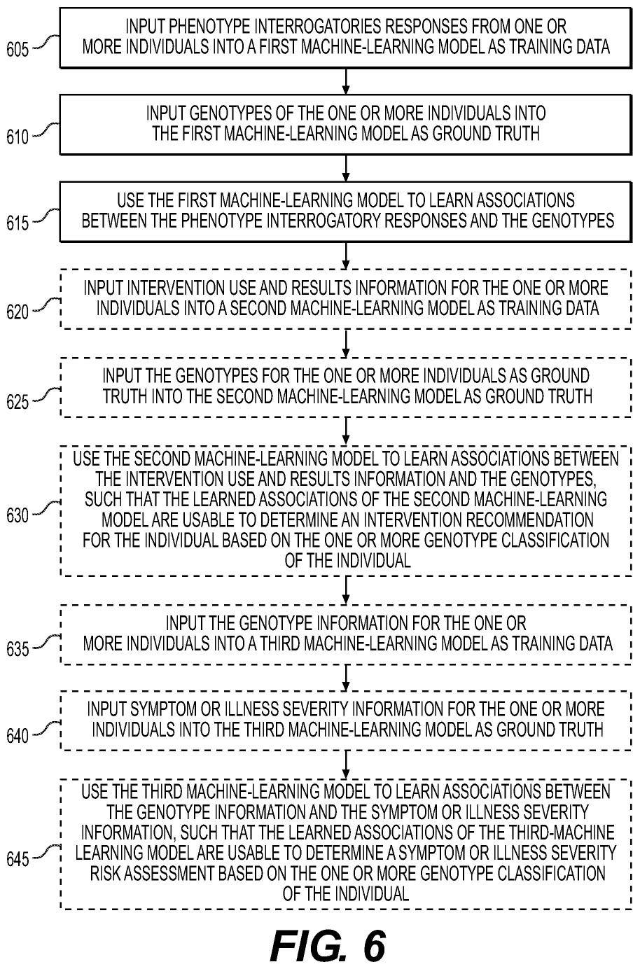

FIG. 6 depicts a flow diagram of an exemplary embodiment of a method of generating a relational model for performing a genotype-based analysis of an individual. At step 605, the assessment system 120, or another system, may input phenotype interrogatories responses from one or more individuals into a first machine-learning model as training data. At step 610, the assessment system 120 may input genotypes of the one or more individuals into the first machine-learning model as ground truth. At step 615, the assessment system 120 may use the first machine-learning model to learn associations between the phenotype interrogatory responses and the genotypes. The learned associations of the first machine-learning model may include one or more different weights or groupings applied to the phenotype interrogatories responses, such that the learned associations of the first machine-learning model are usable to determine one or more genotype classification of an individual based on one or more phenotype interrogatory response from the individual. In some embodiments, the one or more genotype classification that may be determined via the learned associations of the first machine-learning model includes at least one of a genotype for at least one gene of the individual, an indication that the individual has or is at risk for a symptom or illness, or a severity risk assessment of the symptom or illness for the individual

Optionally, at step 620, the assessment system 120 may input intervention use and results information for the one or more individuals into a second machine-learning model as training data. Optionally, at step 625, the assessment system 120 may input the genotypes for the one or more individuals as ground truth into the second machine-learning model as ground truth. Optionally, at step 630, the assessment system 120 may use the second machine-learning model to learn associations between the intervention use and results information and the genotypes, such that the learned associations of the second machine-learning model are usable to determine an intervention recommendation for the individual based on the one or more genotype classification of the individual.

Optionally, at step 635, the assessment system 120 may input the genotype information for the one or more individuals into a third machine-learning model as training data. Optionally, at step 640, the assessment system 120 may input symptom or illness severity information for the one or more individuals into the third machine-learning model as ground truth. Optionally, the assessment system 120 may use the third machine-learning model to learn associations between the genotype information and the symptom or illness severity information, such that the learned associations of the third-machine learning model are usable to determine a symptom or illness severity risk assessment based on the one or more genotype classification of the individual.

Other embodiments of the disclosure will be apparent to those skilled in the art from consideration of the specification and practice of the disclosure disclosed herein. It is intended that the specification and examples be considered as exemplary only.

In general, any process discussed in this disclosure that is understood to be performable by a computer may be performed by one or more processors. Such processes include, but are not limited to: the processes shown in FIGS. 2-4, and the associated language of the specification. The one or more processors may be configured to perform such processes by having access to instructions (computer-readable code) that, when executed by the one or more processors, cause the one or more processors to perform the processes. The one or more processors may be part of a computer system (e.g., one of the computer systems discussed above) that further includes a memory storing the instructions. The instructions also may be stored on a non-transitory computer-readable medium. The non-transitory computer-readable medium may be separate from any processor. Examples of non-transitory computer-readable media include solid-state memories, optical media, and magnetic media.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the disclosed invention requires more features than are expressly recited in any particular embodiment or example. Rather, inventive aspects lie in less than all features of a single foregoing disclosed embodiment. Furthermore, while some embodiments described herein include some but not other features included in other embodiments, combinations of features of different embodiments are meant to be within the scope of the invention, and form different embodiments, as would be understood by those skilled in the art.

Thus, while certain embodiments have been described, those skilled in the art will recognize that other and further modifications may be made thereto without departing from the spirit of the invention, and it is intended to include all such changes and modifications as falling within the scope of this disclosure. For example, functionality may be added or deleted from the block diagrams and operations may be interchanged among functional blocks. Steps may be added or deleted to methods described within the scope of the present disclosure.

We claim:

1. A computer-implemented method of performing a genotype-based analysis of an individual, comprising:
causing an interactive interface of an assessment application operating on a user device to prompt for responses to one or more phenotype interrogatories from an individual;
receiving, from the user device, responses to the one or more phenotype interrogatories from the individual, entered via the interactive interface;
using a relational model, determining at least one genotype classification for the individual based on the received responses to the one or more phenotype interrogatories; and
causing the interactive interface to output information associated with the at least one genotype classification.

2. The computer-implemented method of claim 1, wherein the at least one genotype classification includes at least one of a genotype for at least one gene of the individual, an indication that the individual has or is at risk for a symptom or illness, or a severity risk assessment of the symptom or illness for the individual.

3. The computer-implemented method of claim 2, wherein using the relational model includes:
using a first trained machine-learning model trained, based on (i) training phenotype interrogatories responses from one or more individuals and (ii) ground truth genotypes of the one or more individuals to learn associations between the training phenotype interrogatory responses and the ground truth genotypes, to determine one or more genotypes of the individual based on the received responses and the learned associations.

4. The computer-implemented method of claim 3, wherein the learned associations for the first trained machine-learning model include one or more different weights or groupings applied by the relational model to the responses.

5. The computer-implemented method of claim 3, wherein the information associated with the at least one genotype classification includes one or more of:
a list of one or more symptoms or illnesses for which the individual is at risk; or
an intervention recommendation associated with the one or more symptoms or illnesses.

6. The computer-implemented method of claim 5, wherein:
the information associated with the at least one genotype classification includes the intervention recommendation; and
the method further includes using a second trained machine-learning model trained, based on training intervention use and results information for the one or more individuals and the ground truth genotypes for the one or more individuals to learn associations between the training use and results information and the ground truth genotypes, to determine the intervention recommendation based on the learned associations of the second trained machine-learning model and the one or more genotype classification for the individual.

7. The computer-implemented method of claim 6, wherein:
the at least one genotype classification includes the severity risk assessment; and
the computer-implemented method further includes using a third trained machine-learning model trained, based on training genotypes for the one or more individuals and ground truth symptom or illness severity information for the one or more individuals to learn associations between the training genotypes and the ground truth severity information, to determine the severity risk assessment for the individual based on the learned associations of the third trained machine-learning model and the one or more genotype classification for the individual.

8. The computer-implemented method of claim 1, further comprising:
obtaining additional information associated with the individual, the additional information including one or more of demographic information, location information, lifestyle information, or medical information;
wherein the relational model is configured to further base the at least one genotype classification for the individual on the additional information.

9. The computer-implemented method of claim 8, wherein the additional information is obtained via one or more of:
causing the interactive interface to prompt for response to one or more further interrogatories associated with the additional information;
accessing a profile associated with the individual; or
accessing a database including medical information associated with the individual.

10. The computer-implemented method of claim 9, wherein the one or more phenotype interrogatories includes a plurality of phenotype interrogatories categorized into clusters, each cluster associated with a respective genotype.

11. The computer-implemented method of claim 10, wherein the relational model is configured to assign respective weights to the responses, the respective weights indicative of one or more of a probative value of corresponding phenotype interrogatories to one or more genotypes, or a population prevalence of a phenotype or genotype associated with the corresponding genotype interrogatories.

12. A system for performing a genotype-based analysis of an individual, comprising:
a memory storing instruction and a relational model; and
at least one processor operatively connected to the memory, and configured to execute the instruction to perform operations, including:
causing an interactive interface of an assessment application operating on a user device to prompt for responses to one or more phenotype interrogatories from an individual;
receiving, from the user device, responses to the one or more phenotype interrogatories from the individual, entered via the interactive interface;
using the relational model, determining at least one genotype classification for the individual based on the received responses to the one or more phenotype interrogatories; and
causing the interactive interface to output information associated with the at least one genotype classification.

13. The system of claim 12, wherein the relational model includes a first trained machine-learning model trained, based on (i) training phenotype interrogatories responses from one or more individuals and (ii) ground truth genotypes of the one or more individuals to learn associations between the training phenotype interrogatory responses and the ground truth genotypes, to determine one or more genotypes of the individual based on the received responses and the learned associations.

14. The system of claim 13, wherein the learned associations for the first trained machine-learning model include one or more different weights or groupings applied by the relational model to the responses.

15. The system of claim 14, wherein:
the information associated with the at least one genotype classification includes an intervention recommendation; and
the operations further include using a second trained machine-learning model trained, based on training intervention use and results information for the one or more individuals and the ground truth genotypes for the one or more individuals to learn associations between the training use and results information and the ground truth genotype information, to determine the intervention recommendation based on the learned associations of the second trained machine-learning model and the one or more genotype classification for the individual.

16. The system of claim 15, wherein:
the at least one genotype classification includes a severity risk assessment; and
the operations further include using a third trained machine-learning model trained, based on training genotype information for the one or more individuals and ground truth symptom or illness severity information for the one or more individuals to learn associations between the training genotype information and the ground truth severity information, to determine the severity risk assessment for the individual based on the learned associations of the third trained machine-learning model and the one or more genotype classification for the individual.

17. A method of generating a relational model for performing a genotype-based analysis of an individual, comprising;
inputting phenotype interrogatories responses from one or more individuals into a first machine-learning model as training data;
inputting genotypes of the one or more individuals into the first machine-learning model as ground truth; and
using the first machine-learning model to learn associations between the phenotype interrogatory responses and the genotypes, wherein the learned associations of the first machine-learning model include one or more different weights or groupings applied to the phenotype interrogatories responses, such that the learned associations of the first machine-learning model are usable to determine one or more genotype classification of an individual based on one or more phenotype interrogatory response from the individual.

18. The method of claim 17, wherein the one or more genotype classification includes at least one of a genotype for at least one gene of the individual, an indication that the individual has or is at risk for a symptom or illness, or a severity risk assessment of the symptom or illness for the individual.

19. The method of claim 18, further comprising:
inputting intervention use and results information for the one or more individuals into a second machine-learning model as training data;
inputting the genotypes for the one or more individuals as ground truth into the second machine-learning model as ground truth; and
using the second machine-learning model to learn associations between the intervention use and results information and the genotypes, such that the learned associations of the second machine-learning model are usable to determine an intervention recommendation for the individual based on the one or more genotype classification of the individual.

20. The method of claim 19, further comprising:
inputting the genotype information for the one or more individuals into a third machine-learning model as training data;
inputting symptom or illness severity information for the one or more individuals into the third machine-learning model as ground truth; and
using the third machine-learning model to learn associations between the genotype information and the symptom or illness severity information, such that the learned associations of the third-machine learning model are usable to determine a symptom or illness severity risk assessment based on the one or more genotype classification of the individual.
